(19) United States
(12) Patent Application Publication
(10) Pub. No.: US 2009/0099103 A1
(43) Pub. Date: Apr. 16, 2009

(54) COMBINATIONS OF THERAPEUTIC AGENTS FOR TREATING CANCER

(75) Inventors: Gregory Burke, Randolph, NJ (US); Giorgio Caravatti, Bottmingen (CH); Heidi lane, Biel-Benken (CH); Ronald richard Linnartz, Andover, NJ (US); Richard William Versace, Wanaque, NJ (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG., Basel (CH)

(21) Appl. No.: 12/282,525

(22) PCT Filed: Apr. 4, 2007
(86) PCT No.: PCT/US2007/065911

§ 371 Date: Sep. 11, 2008

Related U.S. Application Data
(60) Provisional application No. 60/789,401, filed on Apr. 5, 2006.

(51) Int. Cl. A61K 31/496; A61K 31/7072; A61K 31/7076; A61K 31/553; A61K 31/7048; A61P 35/00

(52) U.S. Cl. 514/27; 514/252.16; 514/50; 514/46; 514/211.08

(57) ABSTRACT

The invention relates to a combination comprising an Erb-B and VEGF receptor inhibitor; and one or more pharmaceutically active agents; pharmaceutical compositions comprising said combination; methods of treatment comprising said combination; processes for making said combination; and a commercial package comprising said combination.

The invention relates to a combination comprising an Erb-B and vascular endothelial growth factor (VEGF) receptor inhibitor; and one or more pharmaceutically active agents; pharmaceutical compositions comprising said combination; methods of treatment comprising said combination; processes for making said combination; and a commercial package comprising said combination.

### BACKGROUND OF THE INVENTION

7H-Pyrrolo[2,3-d]pyrimidine derivatives exhibit a wide array of biological activities. WO 03/013541 describes 7H-pyrrolo[2,3-d]pyrimidine derivatives, including {6-[4-(4-ethyl-piperazine-1-ylmethyl)-phenyl]-7H-pyrrolo[2,3-d]pyrinidinpyrimidin-4-yl]-((R)-1-phenyl-ethyl)-amine and processes for making. The drug {6-[4-(4-ethyl-piperazin-1-ylmethyl)-phenyl]-7H-pyrrolo[2,3-d]pyrimidin-4-yl}-((R)-1-phenyl-ethyl)-amine is a dual EGF/VEGF inhibitor and exhibits anti-tumour behaviour. However, it is also known that different combinations of active ingredients may increase anti-tumor behaviour. Therefore, there is a continuing need for new combinations of {6-[4-(4-ethyl-piperazin-1-ylmethyl)-phenyl]-7H-pyrrolo[2,3-d]pyrimidin-4-yl}-((R)-1-phenyl-ethyl)-amine.

### SUMMARY OF THE INVENTION

The invention relates to combination which comprises:

(a) an Erb-B and VEGF receptor inhibitor; and

(b) one or more pharmaceutically active agents.

The invention further relates to pharmaceutical compositions comprising:

(a) an Erb-B and VEGF receptor inhibitor;

(b) a pharmaceutically active agent; and

(c) a pharmaceutically acceptable carrier.

The present invention further relates to a commercial package or product comprising:
(a) a pharmaceutical formulation of an Erb-B and VEGF receptor inhibitor; and
(b) a pharmaceutical formulation of a pharmaceutically active agent for simultaneous, concurrent, separate or sequential use.

The combination partners (a) and (b) can be administered together, one after the other or separately in one combined unit dosage form or in two separate unit dosage forms. The unit dosage form may also be a fixed combination.

The present invention further relates to a method of preventing or treating proliferative diseases or diseases that are associated with or triggered by persistent angiogenesis in a mammal, particularly a human, with a combination comprising:

(a) an Erb-B and VEGF receptor inhibitor; and

(b) one or more pharmaceutically active agents.

### BRIEF DESCRIPTION OF THE DRAWINGS

FIG. 1: Shows the percent inhibition for a 81-point 9×9 dose matrix for the combination with {6-[4-(4-ethyl-piperazin-1-ylmethyl)-phenyl]-7H-pyrrolo[2,3-d]pyrimidin-4-yl]-((R)-1-phenyl-ethyl)-amine and N-[1-cyclohexyl-2-oxo-2-(6-phenethyl-octahydro-pyrrolo[2,3-c]pyridin-1-yl-ethyl]-2-methylamino-propionamide in SKOV-3 cells.

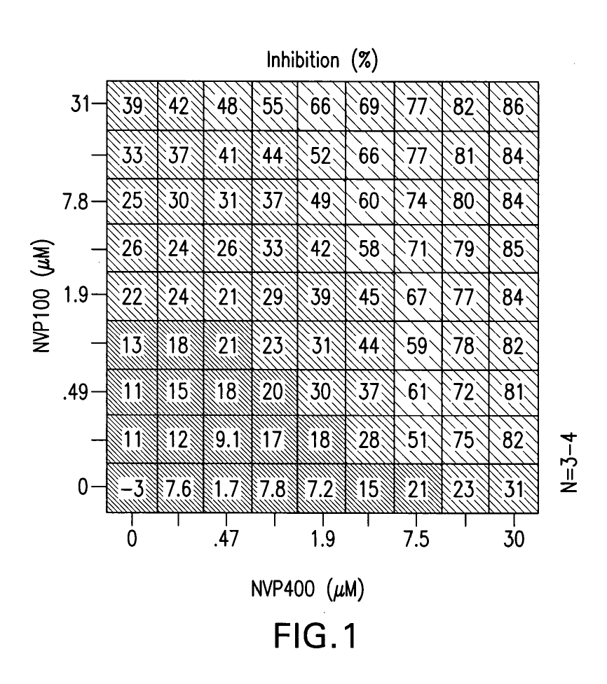

FIG. 2: Shows the synergy for each dose point compared to the Loewe additivity model for the combination with {6-[4-(4-ethyl-piperazin-1-ylmethyl)-phenyl]-7H-pyrrolo[2,3-d]pyrimidin-4-yl]-((R)-1-phenyl-ethyl)-amine and N-[1-cyclohexyl-2-oxo-2-(6-phenethyl-octahydro-pyrrolo[2,3-c]pyridin-1-yl-ethyl]-2-methylamino-propionamide in SKOV-3 cells.

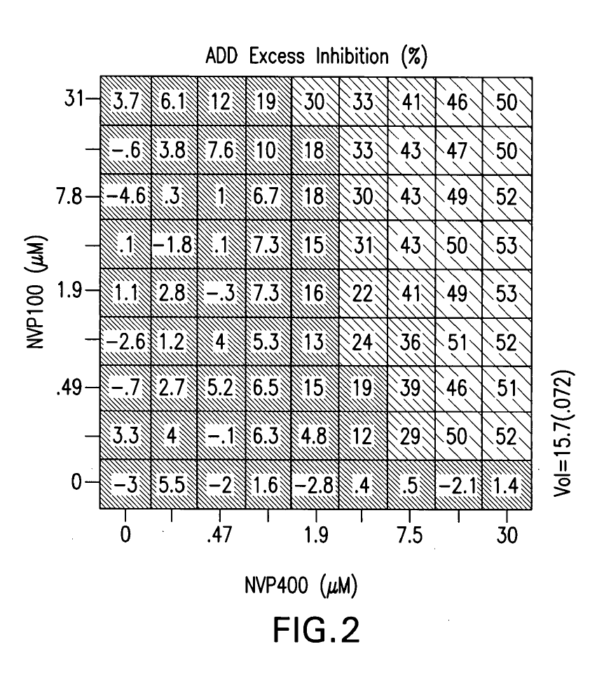

FIG. 3: Shows the isobologram contour at 30% inhibition for the combination with {6-[4-(4-ethyl-piperazin-1-ylmethyl)-phenyl]-7H-pyrrolo[2,3-d]pyrimidin-4-yl]-((R)-1-phenyl-ethyl)-amine and N-[1-cyclohexyl-2-oxo-2-(6-phenethyl-octahydro-pyrrolo[2,3-c]pyridin-1-yl-ethyl]-2-methylamino-propionamide in SKOV-3 cells.

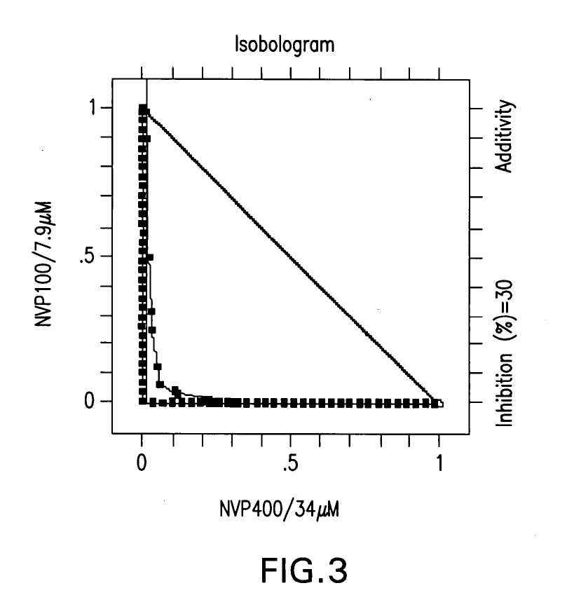

FIG. 4: Shows percent inhibition for a 81-point 9×9 dose matrix for the combination with {6-[4-(4-ethyl-piperazin-1-ylmethyl)-phenyl]-7H-pyrrolo[2,3-d]pyrimidin-4-yl]-((R)-1-phenyl-ethyl)-amine and Cladribine in A549 cells.

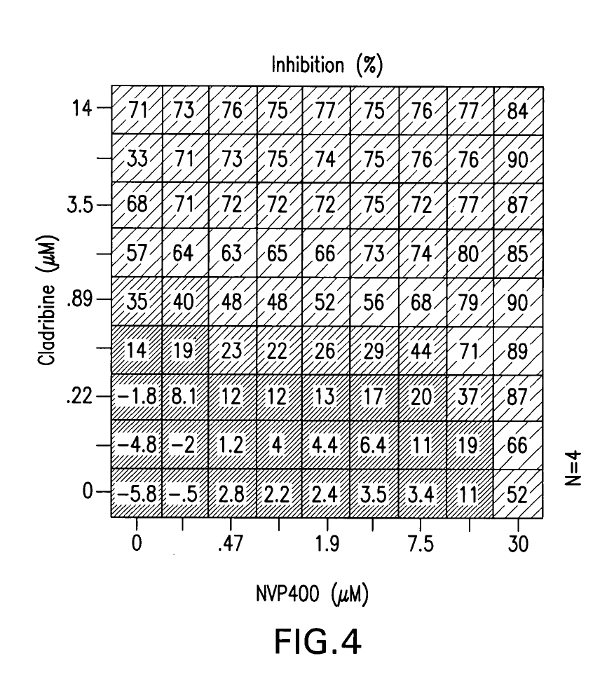

FIG. 5: Shows the synergy for each dose point compared to the Loewe additivity model for the combination with {6-[4-(4-ethyl-piperazin-1-ylmethyl)-phenyl]-7H-pyrrolo[2,3-d]pyrimidin-4-yl]-((R)-1-phenyl-ethyl)-amine and Cladribine in A549 cells.

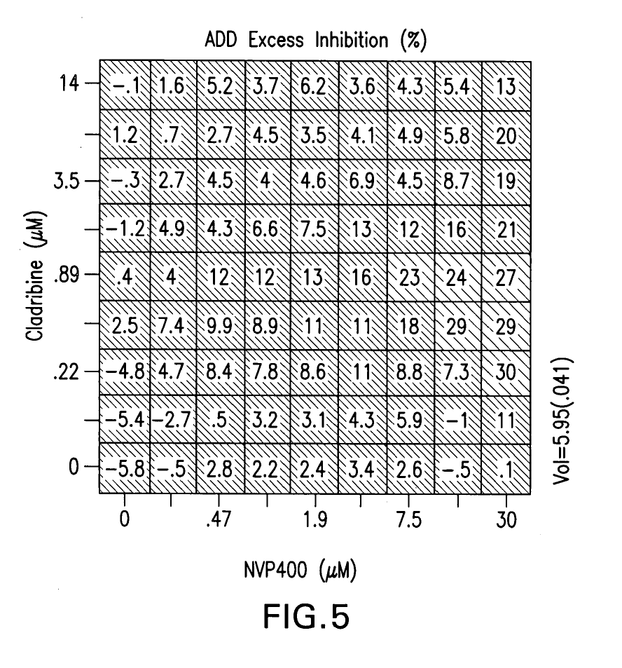

FIG. 6: Shows the isobologram contour at 55% inhibition for the combination with {6-[4-(4-ethyl-piperazin-1-ylmethyl)-phenyl]-7H-pyrrolo[2,3-o]pyrimidin-4-yl]-((R)-1-phenyl-ethyl)-amine and Cladribine in A549 cells.

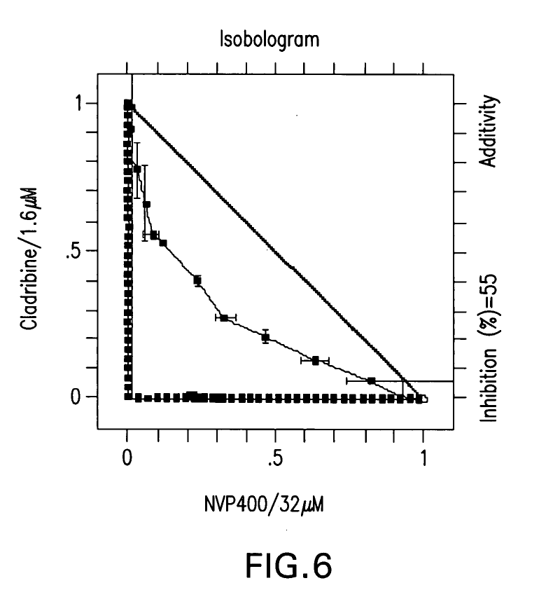

### DETAILED DESCRIPTION OF THE INVENTION

### I. The Erb-B and VEGF Receptor Inhibitor

### Detailed Description of the Inhibitor

The term “an Erb-B and VEGF receptor inhibitor”, as used herein, relates to a compound which targets, decreases or inhibits nucleobase, nucleoside, nucleotide and nucleic acid metabolisms. An example of an adenosine-kinase-inhibitor includes, but is not limited to, 5-Iodotubercidin, which is also known as 7H-pyrrolo[2,3-d]pyrimidin-4-amine, 5-iodo-7-β-D-ribofuranosyl-(9Cl). The compounds of formula (I) have valuable, pharmacologically useful properties. In particular, they exhibit specific inhibitory activities that are of pharmacological interest. They are effective especially as protein tyrosine kinase inhibitors and/or (furthermore) as inhibitors of serine/threonine protein kinases; they exhibit, e.g., powerful inhibition of the tyrosine kinase activity of the epidermal growth factor receptor (EGF-R) and of ErbB-2 kinase. These two protein tyrosine kinase receptors, together with their family members ErbB-3 and ErbB4, play a key role in signal transmission in a large number of mammalian cells, including human cells, especially epithelial cells, cells of the immune system and cells of the central and peripheral nervous system. For example, in various cell types, EGF-induced activation of receptor-associated protein tyrosine kinase is a prerequisite for cell division and hence for the proliferation of the cell population. Most importantly, overexpression of the EGF-R (HER-1) and/or ErbB-2 (HER-2) has been observed in substantial fractions of many human tumours. EGF-R, e.g., was found to be overexpressed in non small-cell lung cancers, squameous carcinoma (head and neck), breast, gastric, ovarian, colon and prostate cancers, as well as in gliomas. ErbB-2 was found to be overexpressed in squameous carcinoma (head and neck), breast, gastric, and ovarian cancers, as well as in gliomas.

In addition to inhibiting the tyrosine kinase activity of the EGF-R, the compounds of formula (I) also inhibit to varying extents other protein tyrosine kinases that are involved in signal transmission mediated by trophic factors, specially the VEGF receptor family (e.g., KDR, Flt-1, Flt-3) but also abl kinase, especially v-abl, kinases from the family of Src, especially c-Src, Lck and Fyn, the other members of the EGF receptor family, such as ErbB-3 (HER-3) and ErbB-4 (HER-4), CSF-1, Kit, FGF receptor and the cyclin-dependent kinases CDK1 and CDK2, all of which play a part in growth regulation and transformation in mammalian cells, including human cells.

The invention relates to 7H-pyrrolo[2,3-d]pyrimidine derivatives of formula (I):

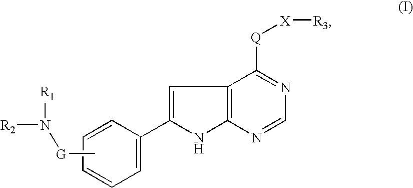

wherein
R₁ and R₂ are each independently of the other hydrogen, unsubstituted or substituted alkyl or cycloalkyl, a heterocyclic radical bonded via a ring carbon atom, or a radical of the formula R₄—Y—(C=Z)-, wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇-alkylene, —C(═O)—, or C₁-C₆-alkylene-C(═O)— wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
   or a salt of the said compounds.

The general terms used hereinbefore and hereinafter preferably have within the context of this disclosure the following meanings, unless otherwise indicated.

Where the plural form is used for compounds, salts and the like, this is taken to mean also a single compound, salt or the like.

Where compounds of formula (I) are mentioned which can form tautomers, it is meant to include also the tautomers of such compounds of formula (I). In particular, tautomerism occurs, e.g., for compounds of formula (I) which contain a 2-hydroxy-pyridyl radical (see, e.g., radical R₃ of the below-mentioned Examples 115-120). In such compounds the 2-hydroxy-pyridyl radical can also be present as pyrid-2(1H)-on-yl.

Asymmetric carbon atoms of a compound of formula I that are optionally present may exist in the (R), (S) or (R,S) configuration, preferably in the (R) or (S) configuration. Substituents at a double bond or a ring may be present in cis- (=Z-) or trans (=E-) form. The compounds may thus be present as mixtures of isomers or preferably as pure isomers.

Preferably alkyl contains up to 20 carbon atoms and is most preferably lower alkyl.

The prefix “lower” denotes a radical having up to and including a maximum of 7, especially up to and including a maximum of 4 carbon atoms, the radicals in question being either unbranched or branched with single or multiple branching.

Lower alkyl is, e.g., methyl, ethyl, n-propyl, isopropyl, n-butyl, isobutyl, sec-butyl, tert-butyl, n-pentyl, isopentyl, neopentyl, n-hexyl or n-heptyl.

Alkyl R₁ and R₂ independently of each other are preferably methyl, ethyl, isopropyl or tert-butyl, especially methyl or ethyl.

Lower alkyl Y is preferably methyl, ethyl or propyl.

Lower alkoxy is for example ethoxy or methoxy, especially methoxy.

Substituted alkyl is preferably lower alkyl as defined above where one or more, preferably one, substituents may be present, such as, e.g., amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Substituted alkyl R₁ and R₂ are independently of each other preferably hydroxy-lower alkyl, N,N-di-lower alkylamino-lower alkyl or morpholinyl-lower alkyl.

Preferably unsubstituted or substituted cycloalkyl R₁ or R₂ contains from 3 up to 20 carbon atoms and is especially unsubstituted or also substituted C₃-C₆-cycloalkyl wherein the substituents are selected from, e.g., unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio, halogen or a heterocyclic radical.

Mono- or disubstituted amino is amino substituted by one or two radicals selected independently of one another from, e.g., unsubstituted or substituted lower alkyl.

Disubstituted amino R₄ is preferably N,N-di-lower alkylamino, especially N,N-dimethylamino or N,N-diethylamino.

A heterocyclic radical contains especially up to 20 carbon atoms and is preferably a saturated or unsaturated monocyclic radical having from 4% or 8-ring members and from 1 to 3 heteroatoms which are preferably selected from nitrogen, oxygen and sulfur, or a bi- or tri-cyclic radical wherein, e.g., one or two carbocyclic radicals, such as, e.g., benzene radicals, are annellated (fused) to the mentioned monocyclic radical. If a heterocyclic radical contains a fused carbocyclic radical then the heterocyclic radical may also be attached to the rest of the molecule of formula (I) via a ring atom of the fused carbocyclic radical. The heterocyclic radical (including the fused carbocyclic radical(s) if present) is optionally substituted by one or more, preferably by one or two, radicals, such as, e.g., unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio or halogen.

Most preferably a heterocyclic radical is pyrrolidinyl, piperidyl, lower alkyl-piperazinyl, di-lower alkyl-piperazinyl, morpholinyl, tetrahydropyranyl, pyridyl, pyridyl substituted by hydroxy or lower alkoxy, or benzodioxolyl, especially pyrrolidinyl, piperidyl, lower alkyl-piperazinyl, di-lower alkyl-piperazinyl or morpholinyl.

A heterocyclic radical R₁ or R₂ is as defined above for a heterocyclic radical with the proviso that it is bonded to the rest of the molecule of formula (I) via a ring carbon atom. Preferably a heterocyclic radical R₁ or R₂ is lower alkyl-piperazinyl or especially preferred tetrahydropyranyl. If one of the two radicals R₁ and R₂ represents a heterocyclic radical, the other is preferably hydrogen.

A heterocyclic radical R₃ is as defined above for a heterocyclic radical with the proviso that it is bonded to Q via a ring carbon atom if X is not present. Preferably a heterocyclic radical R₃ is benzodioxolyl, pyridyl substituted by hydroxy or lower alkoxy, or especially preferred indolyl substituted by halogen and lower alkyl. If R₃ is pyridyl substituted by hydroxy then the hydroxy group is preferably attached to a ring carbon atom adjacent to the ring nitrogen atom.

A heterocyclic radical R₄ is as defined above for a heterocyclic radical and is preferably pyrrolidinyl, piperidyl, lower alkyl-piperazinyl, morpholinyl or pyridyl.

If R₁ and R₂, together with the nitrogen atom to which they are attached, form a heterocyclic radical, the heterocyclic radical is as defined above for a heterocyclic radical and represents preferably pyrrolidinyl, piperidyl, lower alkyl-piperazinyl, di-lower alkyl-piperazinyl or morpholinyl.

An unsubstituted or substituted aromatic radical R₃ has up to 20 carbon atoms and is unsubstituted or substituted, for example in each case unsubstituted or substituted phenyl.

Preferably an unsubstituted aromatic radical R₃ is phenyl. A substituted aromatic radical R₃ is preferably-phenyl substituted by one or more substituents selected independently of one another from the group consisting of unsubstituted or substituted lower alkyl, amino, N-lower alkylamino, N,N-di-lower alkylamino, N-lower alkanoylamino, N,N-di-lower alkanoylamino, hydroxy, lower alkoxy, lower alkanoyl, lower alkanoyloxy, cyano, nitro, carboxy, lower alkoxycarbonyl, carbamoyl, N-lower alkyl-carbamoyl, N,N-di-lower alkyl-carbamoyl, amidino, guanidino, ureido, mercapto, lower alkylthio and halogen. Most preferably a substituted aromatic radical R₃ is phenyl substituted by one or more radicals selected independently of one another from the group consisting of lower alkyl, amino, hydroxy, lower alkoxy, halogen and benzyloxy.

Halogen is primarily fluoro, chloro, bromo or iodo, especially fluoro, chloro or bromo.

C₁-C₇-Alkylene may be branched or unbranched and is, in particular, C₁-C₃-alkylene.

C₁-C₇-Alkylene G is preferably C₁-C₃-alkylene, most preferably methylene (—CH₂—).

If G is not C₁-C₇-alkylene it preferably represents —C(═O)—.

C₁-C₇-Alkylene X is preferably C₁-C₃-alkylene, most preferably methylene (—CH₂—) or ethan-1,1-diyl (—CH(CH₃)—).

Q is preferably —NH—.

Z is preferably oxygen or sulfur, most preferably oxygen.

Salts are especially the pharmaceutically acceptable salts of compounds of formula (I).

Such salts are, formed, e.g., as acid addition salts, preferably with organic or inorganic acids, from compounds of formula (I) with a basic nitrogen atom, especially the pharmaceutically acceptable salts.

In the presence of negatively charged radicals, such as carboxy or sulfo, salts may also be formed with bases, e.g., metal or ammonium salts, such as alkali metal or alkaline earth metal salts, or ammonium salts with ammonia or suitable organic amines, such as tertiary monoamines.

In the presence of a basic group and an acid group in the same molecule, a compound of formula (I) may also form internal salts.

For isolation or purification purposes it is also possible to use pharmaceutically unacceptable salts, e.g., picrates or perchlorates. Only the pharmaceutically acceptable salts or free compounds (if the occasion arises, in the form of pharmaceutical compositions) attain therapeutic use, and these are therefore preferred.

In view of the close relationship between the novel compounds in free form and in the form of their salts, including those salts that can be used as intermediates, e.g., in the purification or identification of the novel compounds, hereinbefore and hereinafter any reference to the free compounds is to be understood as referring also to the corresponding salts, as appropriate and expedient.

Preference is given to a compound of formula (I),

wherein
R₁ and R₂ are each independently of the other hydrogen, unsubstituted or substituted alkyl or cycloalkyl, a heterocyclic radical bonded via a ring carbon atom, or a radical of the formula R₄—Y—(C=Z)-, wherein R₄ is unsubstituted, mono- or di-substituted amino or a heterocyclic radical, Y is either not present or lower alkyl and Z is oxygen or sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇-alkylene;
Q is —NH— or —O—; and
X is either not present or C₁-C₇-alkylene, with the proviso that a heterocyclic radical R₃ is bonded via a ring carbon atom if X is not present;
   or a salt thereof.

Preference is further given to a compound of formula (I), wherein
R₁ and R₂ are each independently of the other hydrogen, unsubstituted or substituted alkyl or cycloalkyl, a heterocyclic radical bonded via a ring carbon atom, or a radical of the formula R₄—Y—(C=Z)-, wherein R₄ is unsubstituted, mono- or di-substituted amino or a heterocyclic radical, Y is either not present or lower alkyl and Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇-alkylene;
Q is —NH—; and
X is either not present or C₁-C₇-alkylene, with the proviso that a heterocyclic radical R₃ is bonded via a ring carbon atom if X is not present;
   or a salt thereof.

Special preference is given to a compound of formula (I),

wherein
R₁ and R₂ are each independently of the other hydrogen, unsubstituted or substituted lower alkyl or C₃-C₆-cycloalkyl, a heterocyclic radical bonded via a ring carbon atom and containing up to 20 carbon atoms, or a radical of the formula R₄—Y—(C=Z)-, wherein R₄ is unsubstituted, mono- or disubstituted amino or a heterocyclic radical containing up to 20 carbon atoms, Y is either not present or lower alkyl and Z is oxygen, with the proviso that R₁ and R₂ are not both hydrogen, or
R₁ and R₂, together with the nitrogen atom to which they are attached form a heterocyclic radical containing up to 20 carbon atoms;
R₃ is a heterocyclic radical containing up to 20 carbon atoms or an unsubstituted or substituted aromatic radical having up to 20 carbon atoms;
G is C₁-C₃-alkylene,
Q is —NH—; and
X is either not present or C₁-C₃-alkylene, with the proviso that a heterocyclic radical R₃ is bonded via a ring carbon atom if X is not present;
   or a salt thereof.

Special preference is further given to a compound of formula (I),

wherein
R₁ and R₂ are each independently of the other hydrogen, lower alkyl, hydroxy-lower alkyl, N,N-di-lower alkylamino-lower alkyl, morpholinyl-lower alkyl, tetrahydropyranyl, or a radical of the formula R₄—Y—(C=Z)-, wherein R₄ is di-lower alkylamino, pyrrolidinyl, piperidyl, lower alkyl-piperazinyl, morpholinyl or pyridyl, Y is either not present or lower alkyl and Z is oxygen, with the proviso that R₁ and R₂ are not both hydrogen, or
R₁ and R₂, together with the nitrogen atom to which they are attached, form a radical selected from the group consisting of pyrrolidinyl, piperidyl, lower alkyl-piperazinyl, di-lower alkyl-piperazinyl and morpholinyl;
R₃ is phenyl, benzodioxolyl, pyridyl substituted by hydroxy or lower alkoxy, indolyl substituted by halogen and lower alkyl, or phenyl substituted by one or more radicals selected independently of one another from the group consisting of lower alkyl, hydroxy, lower alkoxy, halogen and benzyloxy;
G is —CH₂— or —C(═O)—;
Q is —NH— or —O—, with the proviso that Q is —O— if G is —C(═O)—; and
X is either not present, —CH₂— or —CH(CH₃)—, with the proviso that substituted pyridyl or indolyl R₃ is bonded via a ring carbon atom if X is not present;
   or a salt thereof.

Special preference is further also given to a compound of formula (I),

wherein
R₁ and R₂ are each independently of the other hydrogen, lower alkyl, hydroxy-lower alkyl, or a radical of the formula R₄—Y—(C=Z)-, wherein R₄ is di-lower alkylamino, pyrrolidinyl, piperidyl, lower alkyl-piperazinyl, morpholinyl or pyridyl, Y is either not present or lower alkyl and Z is oxygen, with the proviso that R₁ and R₂ are not both hydrogen, or
R₁ and R₂, together with the nitrogen atom to which they are attached, form a radical selected from the group consisting of pyrrolidinyl, piperidyl, lower alkyl-piperazinyl, di-lower alkyl-piperazinyl and morpholinyl;
R₃ is phenyl, benzodioxolyl, pyridyl substituted by hydroxy or lower alkoxy, or phenyl substituted by one or more radicals selected independently of one another from the group consisting of lower alkyl, hydroxy, lower alkoxy, halogen and benzyloxy;
G is —CH₂—;
Q is —NH—; and
X is either not present, —CH₂— or —CH(CH₃)—, with the proviso that substituted pyridyl R₃ is bonded via a ring carbon atom if X is not present;
   or a salt thereof.

Special preference is also given to a compound of formula (I), wherein C₁-C₇-alkylene G is attached to the phenyl ring at position 3 or 4, most especially at position 4.

Very special preference is further given to a compound of formula (I) mentioned in the Examples below, or a salt, especially a pharmaceutically acceptable salt, thereof.

### II. The Pharmaceutically Active Agents

The term “pharmaceutically active agents” is a broad one covering many pharmaceutically active agents having different mechanisms of action. Combinations of some of these with an Erb-B and VEGF receptor inhibitor can result in improvements in cancer therapy. Generally, pharmaceutically active agents are classified according to the mechanism of action. Many of the available agents are anti-metabolites of development pathways of various tumors, or react with the DNA of the tumor cells. There are also agents which inhibit enzymes, such as topoisomerase I and topoisomerase II, or which are antimiotic agents.

By the term “pharmaceutically active agent” is meant especially any pharmaceutically active agent other than an Erb-B and VEGF receptor inhibitor or a derivative thereof. It includes, but is not limited to:
i. an inhibitor of apoptosis proteins (IAPs);
ii. a steroid;
iii. an adenosine-kinase-inhibitor;
iv. an adjuvant;
v. an adrenal cortex antagonist;
vi. AKT pathway inhibitor;
vii. An alkylating agent;
viii. an angiogenesis, inhibitor;
ix. an anti-androgen;
x. an anti-estrogen;
xi. an anti-hypercalcemia agent;
xii. an antimetabolite;
xiii. an apoptosis inducer;
xiv. an aurora kinase inhibitor;
xv. a Bruton's Tyrosine Kinase (BTK) inhibitor;
xvi. a calcineurin inhibitor;
xvii. a CaM kinase II inhibitor;
xviii. a CD45 tyrosine phosphatase inhibitor;
xix. a CDC25 phosphatase inhibitor;
xx. a CHK kinase inhibitor;
xxi. a controlling agent for regulating genistein, olomucine and/or tyrphostins;
xxii. a cyclooxygenase inhibitor;
xxiii. a cRAF kinase inhibitor;
xxiv. a cyclin dependent kinase inhibitor;
xxv. a cysteine protease inhibitor;
xxvi. a DNA intercalator;
xxvii. a DNA strand breaker;
xxviii. an E3 Ligase inhibitor;
xxix. an endocrine hormone;
xxx. compounds targeting, decreasing or inhibiting the activity of the epidermal growth factor family;
xxxi. an EGFR, PDGFR tyrosine kinase inhibitor;
xxxii. a farnesyltransferase inhibitor;
xxxiii. a Flk-1 kinase inhibitor;
xxxiv. a Glycogen synthase kinase-3 (GSK3) inhibitor;
xxxv. a histone deacetylase (HDAC) inhibitor;
xxxvi. a HSP90 inhibitor;
xxxvii. a I-kappa B-alpha kinase inhibitor (IKK);
xxxviii. an insulin receptor tyrosine kinase inhibitor;
xxxix. a c-Jun N-terminal kinase (JNK) kinase inhibitor;
xl. a microtubule binding agent;
xli. a Mitogen-activated protein (MAP) kinase-inhibitor;
xlii. a MDM2 inhibitor;
xliii. a MEK inhibitor;
xliv. a matrix metalloproteinase inhibitor (MMP) inhibitor;
xlv. a NGFR tyrosine-kinase-inhibitor;
xlvi. a p38 MAP kinase inhibitor, including a SAPK2/p38 kinase inhibitor;
xlvii. a p56 tyrosine kinase inhibitor;
xlviii. a PDGFR tyrosine kinase inhibitor;
xlix. a phosphatidylinositol 3-kinase inhibitor;
l. a phosphatase inhibitor;
li. a platinum agent;
lii. a protein phosphatase inhibitor, including a PP1 and PP2 inhibitor and a tyrosine phosphatase inhibitor;
liii. a PKC inhibitor and a PKC delta kinase inhibitor;
liv. a polyamine synthesis inhibitor;
lv. a proteosome inhibitor;
lvi. a PTP1B inhibitor;
lvii. a protein tyrosine kinase inhibitor including a SRC family tyrosine kinase inhibitor; a Syk tyrosine kinase inhibitor; and a JAK-2 and/or JAK-3 tyrosine kinase inhibitor;
lviii. a retinoid;
lix. a RNA polymerase II elongation inhibitor;
lx. a serine/threonine kinase inhibitor;
lxi. a sterol biosynthesis inhibitor;
lxii. a topoisomerase inhibitor; and
lxiii. VEGFR tyrosine kinase inhibitor.

The term “an inhibitor of apoptosis proteins”, as used herein, relates to a compound that inhibits the binding of the Smac protein to IAPs. An example of “an inhibitor of apoptosis protein” includes, but is not limited to, compounds. The present invention relates to compounds of the formula (A):

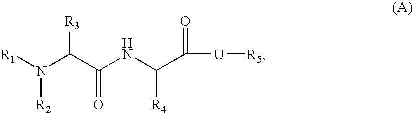

wherein
R₁ is H; C₁-C₄-alkyl; C₁-C₄-alkenyl; C₁-C₄-alkynyl or C₃-C₁₀-cycloalkyl which are unsubstituted or substituted;
R₂ is H; C₁-C₄-alkyl; C₁-C₄-alkenyl; C₁-C₄-alkynyl or C₃-C₁₀-cycloalkyl which are unsubstituted or substituted;
R₃ is H; —CF₃; —C₂F₅; C₁-C₄ alkyl; C₁-C₄-alkenyl; C₁-C₄-alkynyl; —CH₂-Z, or
R₂ and R₃, together with the nitrogen, form a het ring;
Z is H; —OH; F; Cl; —CH₃; —CF₃; —CH₂Cl; —CH₂F or —CH₂OH;
R₄ is C₁-C₁₆-straight or branched alkyl; C₁-C₁₆-alkenyl; C₁-C₁₆-alkynyl; or —C₃-C₁₀-cycloalkyl; —(CH₂)₁₋₆-Z₁; —(CH₂)₀₋₆-arylphenyl; and —(CH₂)₀₋₆-het; wherein alkyl, cycloalkyl and phenyl are unsubstituted or substituted;
Z₁ is —N(R₈)—C(O)—C₁-C₁₀-alkyl; —N(R₈)—C(O)—(CH₂)₁₋₆—C₃-C₇-cycloalkyl; —N(R₈)—C(O)—(CH₂)₀₋₆-phenyl; —N(R₈)—C(O)—(CH₂)₁₋₆-het; —C(O)—N(R₉)(R₁₀); —C(O)—O—C₁-C₁₀-alkyl; —C(O)—O—(CH₂)₁₋₆—C₃-C₇-cycloalkyl; —C(O)—O—(CH₂)₀₋₆-phenyl; —C(O)—O—(CH₂)₁₋₆-het; —O—C(O)—C₁-C₁₀-alkyl; —O—C(O)—(CH₂)₁₋₆—C₃-C₇-cycloalkyl; —O—C(O)—(CH₂)₀₋₆-phenyl; —O—C(O)—(CH₂)₁₋₆-het; wherein alkyl, cycloalkyl and phenyl are unsubstituted or substituted;
het is a 5- to 7-membered heterocyclic ring containing 14 heteroatoms selected from N, O and S, or an 8- to 12-membered fused ring system including at least one 5- to 7-membered heterocyclic ring containing 1, 2 or 3 heteroatoms selected from N, O and S, which heterocyclic ring or fused ring system is unsubstituted or substituted on a carbon or nitrogen atom;
R₈ is H; —CH₃; —CF₃; —CH₂OH or —CH₂Cl;
R₉ and R₁₀ are each independently H; C₁-C₄-alkyl; C₃-C₇-cycloalkyl; —(CH₂)₁₋₆—C₃-C₇-cycloalkyl; —(CH₂)₀₋₆-phenyl; wherein alkyl, cycloalkyl and phenyl are unsubstituted or substituted, or
R₉ and R₁₀, together with the nitrogen, form het;
R₅ is H; C₁-C₁₀-alkyl; aryl; phenyl; C₃-C₇-cycloalkyl; —(CH₂)₁₋₆—C₃-C₇-cycloalkyl; —C₁-C₁₀-alkyl-aryl; —(CH₂)₀₋₆—C₃-C₇-cycloalkyl-(CH₂)₀₋₆-phenyl; —(CH₂)₀₋₄CH—((CH₂)₁₋₄-phenyl)₂; —(CH₂)₀₋₆—CH(phenyl)₂; -indanyl; —C(O)—C₁-C₁₀-alkyl; —C(O)—(CH₂)₁₋₆—C₃-C₇-cycloalkyl; —C(O)—(CH₂)₀₋₆-phenyl; —(CH₂)₀₋₆—C(O)-phenyl; —(CH₂)₀₋₆-het; —C(O)—(CH₂)₁₋₆-het, or
R₅ is a residue of an amino acid, wherein the alkyl, cycloalkyl, phenyl and aryl substituents are unsubstituted or substituted;
U is a as shown in structure (II):

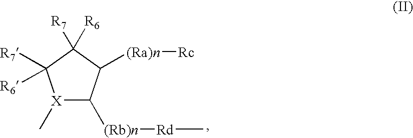

wherein
   n=0-5;
   X is —CH or N;
   Ra and Rb are independently an O, S, or N atom or C₀-C₈-alkyl, wherein one or more of the carbon atoms in the alkyl chain may be replaced by a heteroatom selected from O, S or N, and where the alkyl may be unsubstituted or substituted;
   Rd is selected from:
      (a) —Re-Q-(Rf)ₚ(Rg)_{q} or
      (b) Ar₁-D-Ar₂ or
      (c) Ar₁-D-Ar₂;
   Rc is H, or
   Rc and Rd may together form a cycloalkyl or het; where if Rd and Rc form a cycloalkyl or het, R₅ is attached to the formed ring at a C or N atom;
   p and q are independently 0 or 1;
   Re is C₁-C₈-alkyl or alkylidene;
   Re which may be unsubstituted or substituted;
   Q is N, O, S, S(O) or S(O)₂;
   Ar₁ and Ar₂ are substituted or unsubstituted aryl or het;
   Rf and Rg are each independently none, or H; —C₁-C₁₀-alkyl; C₁-C₁₀-alkylaryl; —OH; —O—C₁-C₁₀-alkyl; —(CH₂)₀₋₆—C₃-C₇-cycloalkyl; —O—(CH₂)₀₋₆-aryl; phenyl; aryl; phenyl-phenyl; —(CH₂)₁₋₈-het; —O—(CH₂)₁₋₆het; —OR₁₁; —C(O)—R₁₂; —C(O)—N(R₁₁)(R₁₂); —N(R₁₁)(R₁₂); —S—R₁₁; —S(O)—R₁₁; —S(O)₂—R₁₁; —S(O)₂—NR₁₁R₁₂; —NR₁₁—S(O)₂—R₁₂; S—C₁-C₁₀-alkyl; aryl-C₁-C₄-alkyl; het-C₁-C₄-alkyl, wherein alkyl, cycloalkyl, het and aryl are unsubstituted or substituted; —SO₂—C₁-C₂-alkyl; —SO₂—C₁-C₂-alkylphenyl; —O—C₁-C₄-alkyl, or
   R_{g}, and R_{f} form a ring selected from het or aryl;
   D is —CO—; —C(O)— or C₁-C₇-alkylene or arylene; —CF₂—; —O—; - or S(O)ₙᵣ, where rn is 0-2; 1,3dioaxolane; or C₁-C₇-alkyl-OH; where alkyl, alkylene or arylene may be unsubstituted or substituted with one or more halogens, OH, —O—C₁-C₆-alkyl, —S—C₁-C₈-alkyl or —CF₃; or D is —N(Rh), wherein Rh is H; C₁-C₇-alkyl (unsubstituted or substituted); aryl; —O(C₁-C₇-cycloalkyl) (unsubstituted or substituted); C(O)—C₀-C₁₀-alkyl; C(O)—C₀-C₁₀-alkyl-aryl; C—O—C₁-C₁₀-alkyl; C—O—C₀-C₁₀-alkyl-aryl or SO₂—CO—C₁₋₁₀-alkyl; SO₂—(C₀-C₁₀-alkylaryl);
   R₆, R₇, R′₆ and R′₇ are each independently H; —C₁-C₁₀-alkyl; —C₁-C₁₀-alkoxy; aryl-C₁-C₁₀-alkoxy; —OH; —O—C₁-C₁₀-alkyl; —(CH₂)₀₋₆—C₃-C₇-cycloalkyl; —O—(CH₂)₀₋₆-aryl; phenyl; —(CH₂)₁₋₆-het; —O—(CH₂)₁₋₆-het; —OR₁₁; —C(O)—R₁₁; —C(O)—N(R₁₁)(R₁₂); —N(R₁₁)(R₁₂); —S—R₁₁; —S(O)—R₁₁; —S(O)₂—R₁₁; —S(O)₂—NR₁₁R₁₂; —NR₁₁, —S(O)₂—R₁₂, wherein alkyl, cycloalkyl and aryl are unsubstituted or substituted; and R₆, R₇, R₁₈ and R₁₇ can be united to form a ring system;
R₁₁, and R₁₂ are independently H; C₁-C₁₀-alkyl; —(CH₂)₀₋₆—C₃-C₇-cycloalkyl; —(CH₂)₀₋₆—(CH)₀₋₁(aryl)₁₋₂; —C(O)—C₁-C₁₀-alkyl; —C(O)—(CH₂)₁₋₆—C₃-C₇-cycloalkyl; —C(O)—O—(CH₂)₀₋₆-aryl; —C(O)—(CH₂)₀₋₈—O-fluorenyl; —C(O)—NH—(CH₂)₀₋₆-aryl; —C(O)—(CH₂)₀₋₆-aryl; —C(O)—(CH₂)₁₄-het; —C(S)—C₁-C₁₀-alkyl; —C(S)—(CH₂)₁₋₆—C₃-C₇-cycloalkyl; —C(S)—O—(CH₂)₀₋₆-aryl; —C(S)—(CH₂)₀₋₆—O-fluorenyl; —C(S)—NH—(CH₂)₀₋₆-aryl; —C(S)—(CH₂)₀₋₆-aryl; —C(S)—(CH₂)₁₋₆-het, wherein alkyl, cycloalkyl and aryl are unsubstituted or substituted, or
   R₁₁, and R₁₂ are a substituent that facilitates transport of the molecule across a cell membrane, or
   R₁₁, and R₁₂, together with the nitrogen atom, form het;
      wherein the alkyl substituents of R₁₁ and R₁₂ may be unsubstituted or substituted by one or more substituents selected from C₁-C₁₀-alkyl, halogen, OH, —O—C₁-C₆-alkyl, —S—C₁-C₆-alkyl or —CF₃;
      substituted cycloalkyl substituents of R₁₁ and R₁₂ are substituted by one or more substituents selected from a C₁-C₁₀-alkene; C₁-C₆-alkyl; halogen; OH; —O—C₁-C₆-alkyl; —S—C₁-C₆-alkyl or —CF₃; and
      substituted phenyl or aryl of R₁₁, and R₁₂ are substituted by one or more substituents selected from halogen; hydroxy; C₁-C₄-alkyl; C₁-C₄-alkoxy; nitro; —CN; —O—C(O)—C₁-C₄-alkyl and —C(O)—O—C₁-C₄-aryl,
         or pharmaceutically acceptable salts thereof.

“Aryl” is an aromatic radical having 6-14 carbon atoms, which may be fused or unfused, and which is unsubstituted or substituted by one or more, preferably one or two substituents, wherein the substituents are as described below. Preferred “aryl” is phenyl, naphthyl or indanyl.

“Het” refers to heteroaryl and heterocyclic rings and fused rings containing aromatic and non-aromatic heterocyclic rings. “Het” is a 5- to 7-membered heterocyclic ring containing 1-4 heteroatoms selected from N, O and S, or an 8- to 12-membered fused ring system including at least one 5- to 7-membered heterocyclic ring containing 1, 2 or 3 heteroatoms selected from N, O and S. Suitable het substituents include unsubstituted and substituted pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, 1,4-oxathiapane, furyl, thienyl, pyrrole, pyrazole, triazole, 1,2,3-triazole, tetrazolyl, oxadiazole, thiophene, imidazol, pyrrolidine, pyrrolidone, thiazole, oxazole, pyridine, pyrimidine, isoxazolyl, pyrazine, quinoline, isoquinoline, pyridopyrazine, pyrrolopyridine, furopyridine, indole, benzofuran, benzothiofuran, benzindole, benzoxazole, pyrroloquinoline, and the like. The het substituents are unsubstituted or substituted on a carbon atom by halogen, especially fluorine or chlorine, hydroxy, C₁-C₄-alkyl, such as methyl and ethyl, C₁-C₄-alkoxy, especially methoxy and ethoxy, nitro, —O—C(O)—C₁-C₄-alkyl or —C(O)—O—C₁-C₄-alkyl or on a nitrogen by C₁-C₄-alkyl, especially methyl or ethyl, —O—C(O)—C₁-C₄-alkyl or —C(O)—O—C₁-C₄-alkyl, such as carbomethoxy or carboethoxy.

When two substituents together with a commonly bound nitrogen are het, it is understood that the resulting heterocyclic ring is a nitrogen-containing ring, such as aziridine, azetidine, azole, piperidine, piperazine, morphiline, pyrrole, pyrazole, thiazole, oxazole, pyridine, pyrimidine, isoxazolyl and the like.

Halogen is fluorine, chlorine, bromine or iodine, especially fluorine and chlorine.

Unless otherwise specified “alkyl” includes straight or branched chain alkyl, such as methyl, ethyl, n-propyl, isopropyl, n-butyl, sec-butyl, tert-butyl, n-pentyl and branched pentyl, n-hexyl and branched hexyl and the like.

A “cycloalkyl” group means C₃-C₁₀-cycloalkyl having 3- to 8-ring carbon atoms and may be, e.g., cyclopropyl, cyclobutyl, cyclopentyl, cyclohexyl, cycloheptyl or cyclooctyl. Preferably, cycloalkyl is cycloheptyl. The cycloalkyl group may be unsubstituted or substituted with any of the substituents defined below, preferably halo, hydroxy or C₁-C₄-alkyl, such as methyl. Preferred compounds of formula (I) are:
N-[1-Cyclohexyl-2-oxo-2-(6-phenethyl-octahydro-pyrrolo[2,3-c]pyridin-1-yl)-ethyl]-2-methylamino-acetamide;
2-Methylamino-N-[2-methyl-1-(7-oxo-6-phenethyl-octahydro-pyrrolo[2,3-c]pyridine-1-carbonyl)-propyl]-propionamide;
2-Methylamino-N-[2-methyl-1-(7-oxo-6-phenethyl-octahydro-pyrrolo[2,3-c]pyridine-1-carbonyl)-propyl]-propionamide;
2-Methylamino-N-[2-methyl-1-(8-oxo-7-phenethyl-octahydro-pyrrolo[2,3-c]azepine-1-carbonyl)-propyl]-propionamide;
2-Methylamino-N-[2-methyl-1-(7-oxo-6-phenethyl-octahydro-pyrrolo[2,3-c]pyridine-1-carbonyl)-propyl]-butyramide;
2-Methylamino-N-[2-methyl-1-(7-oxo-6-phenethyl-octahydro-pyrrolo[2,3-c]pyridine-1-carbonyl)-propyl]-butyramide;
2-Methylamino-N-[2-methyl-1-(8-oxo-7-phenethyl-octahydro-pyrrolo[2,3-c]azepine-1-carbonyl)-propyl]-butyramide;
N-[1-Cyclohexyl-2-oxo-2-(7-oxo-6-phenethyl-octahydro-pyrrolo[2,3-c]pyridin-1-yl)-ethyl]-2-methylamino-propionamide;
2-Methylamino-N-{2-methyl-1-[5-(3-methyl-hexa-3,5-dienyl)-6-oxo-hexahydro-pyrrolo[3,4-b]pyrrole-1-carbonyl]-propyl}-propionamide;
2-Methylamino-N-[2-methyl-1-(3-methyl-7-oxo-6-phenethyl-octahydro-pyrrolo[2,3-c]pyridine-1-carbonyl)-propyl]-propionamide:
2-Methylamino-N-[2-methyl-1-(3-methyl-7-oxo-6-phenethyl-octahydro-pyrrolo[2,3-c]pyridine-1-carbonyl)-propyl]-propionamide;
N-[1-(4-Benzyloxy-7-oxo-6-phenethyl-octahydro-pyrrolo[2,3-c]pyridine-1-carbonyl)-2-methyl-propyl]-2-methylamino-propionamide;
N-[1-Cyclohexyl-2-oxo-2-(8-oxo-7-phenethyl-octahydro-pyrrolo[2,3-c]azepin-1-yl)-ethyl]-2-methylamino-butyramide;
N-[1-Cyclohexyl-2-oxo-2-(8-oxo-7-phenethyl-octahydro-pyrrolo[2,3-c]azepin-1-yl)ethyl]-2-methylamino-butyramide;
N-[1-Cyclohexyl-2-oxo-2-(7-phenethyl-octahydro-pyrrolo[2,3-c]azepin-1-yl)-ethyl]-2-methylamino-propionamide;
2-Methylamino-N-[2-methyl-1-(8-oxo-7-phenethyl-octahydro-pyrrolo[2,3-c]azepine-1-carbonyl)-propyl]-butyramide;
(S)—N—{(S)-2-[(R)-2-(3-Benzyl-phenyl)-pyrrolidin-1-yl]-1-cyclohexyl-2-oxo-ethyl}-2-methylamino-propionamide;
(S)—N—{(S)-2-[(S)-2-(3-Benzyl-phenyl)-pyrrolidin-1-yl]-1-cyclohexyl-2-oxo-ethyl}-2-methylamino-propionamide;
(S)-2-Methylamino-N—((S)-2-methyl-1-{(S)-2-[3-(methyl-phenyl-amino)-phenyl]-pyrrolidine-1-carbonyl}-propyl)-propionamide;
(S)—N—((S)-1-Cyclohexyl-2-{(S)-2-[3-(methyl-phenyl-amino)-phenyl]-pyrrolidin-1-yl}-2-oxo-ethyl)-2-methylamino-propionamide;
(S)—N—((S)-1-Cyclohexyl-2-{(R)-2-[3-(methyl-phenyl-amino)-phenyl]-pyrrolidin-1-yl}-2-oxo-ethyl)-2-methylamino-propionamide;
(S)—N—{(S)-1-Cyclohexyl-2-oxo-2-[(R)-2-(3-phenoxy-phenyl)-pyrrolidin-1-yl]-ethyl}-2-methylamino-propionamide;
(S)—N—{(S)-1-Cyclohexyl-2-oxo-2-[(S)-2-(3-phenoxy-phenyl)-pyrrolidin-1-yl]-ethyl}-2-methylamino-propionamide;
(S)—N—{(S)-1-Cyclohexyl-2-oxo-2-[(R)-2-(3-phenylsulfanyl-phenyl)-pyrrolidin-1-yl]-ethyl}-2-methylamino-propionamide;
(S)—N—{(S)-1-Cyclohexyl-2-oxo-2-[(S)-2-(3-phenylsulfanyl-phenyl)-pyrrolidin-1-yl]-ethyl}-2-methylamino-propionamide;
(S)—N—{(S)-2-[(R)-2-(3-Benzenesulfonyl-phenyl)-pyrrolidin-1-yl]-1-cyclohexyl-2-oxo-ethyl}-2-methylamino-propionamide;
(S)—N—{(S)-2-[(S)-2-(2-Benzyl-2H-tetrazol-5-yl)-pyrrolidin-1-yl]-1-cyclohexyl-2-oxo-ethyl}-2-methylamino-propionamide;
(S)—N—{(S)-2-[(S)-2-(2-Benzyl-2H-tetrazol-5-yl)-pyrrolidin-1-yl]-1-cyclohexyl-2-oxo-ethyl}-2-methylamino-butyramide;
(S)—N—{(S)-2-[(S)-2-(1-Benzyl-1H-tetrazol-5-yl)-pyrrolidin-1-yl]-1-cyclohexyl-2-oxo-ethyl}-2-methylamino-propionamide;
(S)—N—{(S)-2-[(S)-2-(1-Benzyl-1H-tetrazol-5-yl)-pyrrolidin-1-yl]-1-cyclohexyl-2-oxo-ethyl}-2-methylamino-butyramide;
(S)—N—{(S)-2-[2-(Benzyloxyimino-yl)-pyrrolidin-1-yl]-1-cyclohexyl-2-oxo-ethyl}-2-methylamino-propionamide;
(S)-2-Methylamino-N—{(S)-2-methyl-1-[2-((S)-phenylmethanesulfonylamino-methyl)-pyrrolidine-1-carbonyl]-propyl}-propionamide;
(S)-2-Methylamino-N—{(S)-2-methyl-1-[2-((S)-phenylmethanesulfonylamino-methyl)-pyrrolidine-1-carbonyl]-propyl}-butyramide;
N-(1-Cyclohexyl-2-{(S)-2-[(ethyl-indan-2-yl-amino)-methyl]-pyrrolidin-1-yl}-2-oxo-ethyl)-2-((S)-methylamino)-propionamide;
(S)—N—[(S)-1-Cyclohexyl-2-(2{[(S)-indan-2-yl-(2,2,2-trifluoro-ethyl)-amino]-methyl}-pyrrolidin-1-yl)-2-oxo-ethyl]-2-methylamino-propionamide;
(S)—N—((S)-1-Cyclohexyl-2-{2-[((S)-cyclohexyl-phenethyl-amino)-methyl]-pyrrolidin-1-yl}-2-oxo-ethyl)-2-methylamino-propionamide;
(S)—N—((S)-2-{2-[((S)-tert-Butyl-phenethyl-amino)-methyl]-pyrrolidin-1-yl}-1-cyclohexyl-2-oxo-ethyl)-2-methylamino-propionamide;
(S)—N—((S)-1-Cyclohexyl-2-{2-[((S)-furan-2-ylmethyl-phenethyl-amino)-methyl]-pyrrolidin-1-yl}-2-oxo-ethyl)-2-methylamino-propionamide;
(S)—N—[(S)-1-Cyclohexyl-2-oxo-2-{[(2-[(S)-phenethyl-(4-phenyl-butyl)-amino]-methyl}-pyrrolidin-1-yl)-ethyl]-2-methylamino-propionamide;
(S)—N—[(S)-1-Cyclohexyl-2-(2-{[(S)-methyl-(4-phenyl-butyl)-amino]-methyl}-pyrrolidin-1-yl)-2-oxo-ethyl]-2-methylamino-propionamide;
N—[(S)-1-(S)-Cyclohexyl-2-oxo-2-((R)-6-phenethyl-octahydro-pyrrolo[2,3-c]pyridin-1-yl)-ethyl]-acetamide;
(S)—N—[(S)— 1-(S)-Cyclohexyl-2-oxo-2-((R)-6-phenethyl-octahydro-pyrrolo[2,3-c]pyridin-1-yl)-ethyl]-2-methylamino-butyramide;
(S)-2-Methylamino-N—[(S)-2-methyl-1-((R)-6-phenethyl-octahydro-pyrrolo[2,3-c]pyridine-1-carbonyl)-propyl]-propionamide;
(S)—N—[(S)-2,2-Dimethyl-1-((R)-6-phenethyl-octahydro-pyrrolo[2,3-c]pyridine-1-carbonyl)-propyl]-2-methylamino-propionamide;
(S)-2-Methylamino-N—[(S)-2-methyl-1-((R)-6-phenethyl-octahydro-pyrrolo[2,3-c]pyridine-1-carbonyl)-propyl]-butyramide;
(S)—N—[(S)-2,2-Dimethyl-1-((3aR,7aS)-6-phenethyl-octahydro-pyrrolo[2,3-c]pyridine-1-carbonyl)-propyl]-2-methylamino-propionamide;
(S)—N—((S)-1-Cyclohexyl-2-oxo-2-{(3aR,7aS)-6-[2-(2-trifluoromethoxy-phenyl)-ethyl]-octahydro-pyrrolo[2,3-c]pyridin-1-yl}-ethyl)-2-methylamino-propionamide;
(S)—N—((S)-1-Cyclohexyl-2-oxo-2-{(3aR,7aS)-6-[2-(3-trifluoromethoxy-phenyl)-ethyl]-octahydro-pyrrolo[2,3-c]pyridin-1-yl}-ethyl)-2-methylamino-propionamide;
(S)—N—[(S)-1-Cyclohexyl-2-oxo-2-((3aR,6aR)-5-phenethyl-hexahydro-pyrrolo[3,4-b]pyrrol-1-yl)-ethyl]-2-methylamino-butyramide;
(S)—N—[(S)-1-Cyclohexyl-2-oxo-2-((3aS,6aS)-5-phenethyl-hexahydro-pyrrolo[3,4-b]pyrrol-1-yl)-ethyl)-2-methylamino-butyramide;
(S)—N—[(S)-1-Cyclohexyl-2-oxo-2-((3aS,6aS)-5-phenethyl-hexahydro-pyrrolo[3,4-b]pyrrol-1-yl)-ethyl]-2-methylamino-propionamide;
(S)—N—[(S)-1-Cyclohexyl-2-oxo-2-((3aS,6aS)-6-oxo-5-phenethyl-hexahydro-pyrrolo[3,4-b]pyrrol-1-yl)-ethyl]-2-methylamino-butyramide;
(S)—N—[(R)-1-Cyclohexyl-2-oxo-2-((3aS,6aS)-6-oxo-5-phenethyl-hexahydro-pyrrolo[3,4-b]pyrrol-1-yl)-ethyl]-2-methylamino-butyramide;
(S)—N—[(S)-1-Cyclohexyl-2-oxo-2-((3aS,6aS)-6-oxo-5-phenethyl-hexahydro-pyrrolo[3,4-b]pyrrol-1-yl)-ethyl-2-methylamino-propionamide;
(S)—N—[(R)-1-Cyclohexyl-2-oxo-2-((3aS,6aS)-6-oxo-5-phenethyl-hexahydro-pyrrolo[3,4-b]pyrrol-1-yl)-ethyl]-2-methylamino-propionamide;
(S)—N—[(S)-1-(R)-Cyclohexyl-2-oxo-2-((S)-7-phenethyl-octahydro-pyrrolo[2,3-c]azepin-1-yl)-ethyl]-2-methylamino-propionamide;
(S)—N—[(S)-1-(S)-Cyclohexyl-2-oxo-2-((R)-8-oxo-7-phenethyl-octahydro-pyrrolo[2,3-c]azepin-1-yl)-ethyl]-2-methylamino-butyramide;
N-[1-cyclohexyl-2-oxo-2-(6-phenethyl-octahydro-pyrrolo[2,3-c]pyridin-1-yl)-ethyl]-2-methylamino-propionamide;
N-{1-cyclohexyl-2-oxo-2-(2-(3-phenoxy-phenyl)pyrrolidin-1-yl]-ethyl}-2-methylamino-propionamide;
N-[1-cyclohexyl-2-oxo-2-(7-phenethyl-octahydro-pyrrolo[2,3-c]azepin-1-yl)-ethyl]-2-methylaminopropionamide;
(S)—N—((S)-1-Cyclohexyl-2-{(2S,3R)-2-[(ethyl-phenethyl-amino)-methyl]-3-methyl-pyrrolidin-1-yl}-2-oxo-ethyl)-2-methylamino-propionamide;
N-{2-[2-(2-Benzyl-2H-tetrazol-5-yl)-pyrrolidin-1-yl]-cyclohexyl-2-oxo-ethyl}-2-methylamino-butyramide;
N-{2-[2-Benxyloxyimino-methyl)-pyrrolidin-1-yl}-1-cyclohexyl-2-oxo-ethyl-2-methylamino-propionamide;
(S)—N—{(S)-1-Cyclohexyl-2-oxo-2-[(S)-2-(3-phenoxy-phenyl)-pyrrolidin-1-yl]-ethyl}-2-methylamino-propionamide;
(S)—N—{(S)-1-Cyclohexyl-2-oxo-2-[(S)-2-(3-phenylsulfanyl-phenyl)-pyrrolidin-1-yl]-ethyl}-2-methylamino-propionamide;
(S)—N—{(S)-2-[(S)-2-(2-Benzyl-2H-tetrazol-5-yl)-pyrrolidin-1-yl]-1-cyclohexyl-2-oxo-ethyl}-2-methylamino-propionamide;
(S)—N—{(S)-2-[(S)-2-(2-Benzyl-2H-tetrazol-5-yl)-pyrrolidin-1-yl]-1-cyclohexyl-2-oxo-ethyl}-2-methylamino-butyramide;
(S)—N—{(S)-2-[(S)-2-(1-Benzyl-1H-tetrazol-5-yl)-pyrrolidin-1-yl]-1-cyclohexyl-2-oxo-ethyl}-2-methylamino-propionamide; and
(S)—N—{(S)-2-[(S)-2-(1-Benzyl-1H-tetrazol-5-yl)-pyrrolidin-1-yl]-1-cyclohexyl-2-oxo-ethyl}-2-methylamino-butyramide;
   and pharmaceutically acceptable salts thereof.

A preferred compounds within the scope of formula (I) is N-(1-cyclohexyl-2-oxo-2-(6-phenethyl-octahydro-pyrrolo[2,3-c]pyridin-1-yl-ethyl]-2-methylamino-propionamide of formula (III):

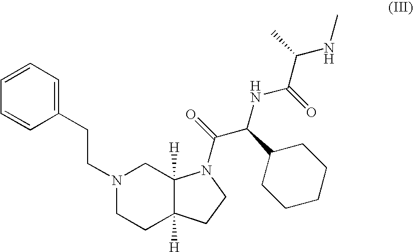

The term “a steroid”, as used herein, relates to prednisone.

The term “an adenosine-kinase-inhibitor”, as used herein, relates to a compound which targets, decreases or inhibits nucleobase, nucleoside, nucleotide and nucleic acid metabolisms. An example of an adenosine-kinase-inhibitor includes, but is not limited to, 5-Iodotubercidin, which is also known as 7H-pyrrolo[2,3-d]pyrimidin-4-amine, 5-iodo-7-β-D-ribofuranosyl-(9Cl).

The term “an adjuvant”, as used herein, refers to a compound which enhances the 5-FU-TS bond as well as a compound which targets, decreases or inhibits, alkaline phosphatase. Examples of an adjuvant include, but are not limited to, Leucovorin, and Levamisole.

The term “an adrenal cortex antagonist”, as used herein, relates to a compound which targets, decreases or inhibits the activity of the adrenal cortex and changes the peripheral metabolism of corticosteroids, resulting in a decrease in 17-hydroxycorticosteroids. An example of an adrenal cortex antagonist includes, but is not limited to, Mitotane.

The term “AKT pathway inhibitor”, as used herein, relates to a compound which targets, decreases or inhibits cell proliferation. AKT, also known as protein kinase B (PKB), a serine/threonine kinase, is a critical enzyme in several signal transduction pathways involved in diabetes. The principal role of AKT in the cell is to facilitate growth factor-mediated cell survival and to block apoptotic cell death. A target of the AKT pathway inhibitor includes, but is not limited to, Pi3K/AKT. Examples of an AKT pathway inhibitor, include, but are not limited to, Deguelin, which is also known as 3H-bis[1]benzopyrano[3,4-b:6′,5′-e]pyran-7(7aH)-one, 13,13a-dihydro-9,10-dimethoxy-3,3-dimethyl-, (7aS,13aS)-(9Cl); and Trciribine, which is also known as 1,4,5,6,8-pentaazaacenaphthylen-3-amine, 1,5-dihydro-5-methyl-1-β-D-ribofuranosyl-(9Cl).

The term “an alkylating agent”, as used herein, relates to a compound which causes alkylation of DNA and results in breaks in the DNA molecules as well as cross-linking of the twin strands, thus interfering with DNA replication and transcription of RNA. Examples of an alkylating agent include, but are not limited to, Chlorambucil, cyclophosphamide, Dacarbazine, Lomustine, Procarbazine, Thiotepa, Melphalan, Temozolomide (TEMODAR), Carmustine, Ifosfamide, Mitomycin, Altretamine, Busulfan, Machlorethamine hydrochloride, nitrosourea (BCNU or Gliadel), Streptozocin and estramustine. Cyclophosphamide can be administered, e.g., in the form as it is marketed, e.g., under the trademark CYCLOSTIN; and ifosfamide as HOLOXAN.

The term “an angiogenesis inhibitor”, as used herein, relates to a compound which targets, decreases or inhibits the production of new blood vessels. Targets of an angiogenesis inhibitor include, but are not limited to, methionine aminopeptidase-2 (MetAP-2), macrophage inflammatory protein-1 (MIP-1 alpha), CCL5, TGF-beta, lipoxygenase, cyclooxygenase and topoisomerase. Indirect targets of an angiogenesis inhibitor include, but are not limited to, p21, p53, CDK2, and collagen synthesis. Examples of an angiogenesis inhibitor include, but are not limited to, Fumagillin, which is known as 2,4,6,8-Decatetraenedioic acid, mono[(3R,4S,5S,6R)-5-methoxy-4-[(2R,3R)-2-methyl-3-(3-methyl-2-butenyl)oxiranyl]-1-oxaspiro[2.5]oct-6-yl]ester, (2E,4E,6E,8E)-(9Cl); Shikonin, which is also known as 1,4-naphthalenedione, 5,8-dihydroxy-2-[(1R)-1-hydroxy-4-methyl-3-pentenyl]-(9Cl); Tranilast, which is also known as benzoic acid, 2-[[3-(3,4-dimethoxyphenyl)-1-oxo-2-propenyl]amino]-(9Cl); ursolic acid; suramin; and thalidomide.

The term “an anti-androgen”, as used herein, relates to a compound which blocks the action of androgens of adrenal and testicular origin which stimulate the growth of normal and malignant prostatic tissue. Examples of an anti-androgen include, but are not limited to, Nilutamide; bicalutamide (CASODEX), which can be formulated, e.g., as disclosed in U.S. Pat. No. 4,636,505.

The term “an anti-estrogen”, as used herein, relates to a compound which antagonizes the effect of estrogens at the estrogen receptor level. Examples of an anti-estrogen include, but are not limited to, Toremifene; Letrozole; Testolactone; Anastrozole; Bicalutamide; Flutamide; Tamoxifen Citrate; Exemestane; Fulestrant; tamoxifen; fulvestrant; raloxifene and raloxifene hydrochloride. Tamoxifen can be administered in the form as it is marketed, e.g., NOLVADEX; and raloxifene hydrochloride is marketed as EVISTA. Fulvestrant can be formulated as disclosed in U.S. Pat. No. 4,659,516 and is marketed as FASLODEX. A combination of the invention comprising a pharmaceutically active agent which is an anti-estrogen is particularly useful for the treatment of estrogen receptor positive tumors, e.g., breast tumors.

The term “an anti-hypercalcemia agent”, as used herein, refers to compounds which are used to treat hypercalcemia. Examples of an anti-hypercalcemia agent include, but are not limited to, gallium (III) nitrate hydrate; and pamidronate disodium.

The term “antimetabolite”, as used herein, relates to a compound which inhibits or disrupts the synthesis of DNA resulting in cell death. Examples of an antimetabolite include, but are not limited to, 6-mercaptopurine; Cytarabine; Fludarabine; floxuridine, Flexuridine; Fluorouracil; Capecitabine; Raltitrexed; Methotrexate; Cladribine; Gemcitabine; Gemcitabine hydrochloride; Thioguanine; Hydroxyurea; DNA de-methylating agents, such as 5-azacytidine and decitabine; edatrexate; and folic acid antagonists such as, but not limited to, pemetrexed. Capecitabine can be administered, e.g., in the form as it is marketed, e.g., under the trademark XELODA; and gemcitabine as GEMZAR.

The term “an apoptosis inducer”, as used herein, relates to a compound which induces the normal series of events in a cell that leads to its death. The apoptosis inducer of the present invention may selectively induce the X-linked mammalian inhibitor of apoptosis protein XIAP. The apoptosis inducer of the present invention may downregulate BCL-xL. Examples of an apoptosis inducer include, but are not limited to, ethanol, 2-[[3-(2,3-dichlorophenoxy)propyl]amino]-(9Cl); gambogic acid; Embelin, which is also known as 2,5-cyclohexadiene-1,4-dione, 2,5-dihydroxy-3-undecyl-(9Cl); and Arsenic Trioxide.

The term “an aurora kinase inhibitor”, as used herein, relates to a compound which targets, decreases or inhibits later stages of the cell cycle from the G2/M check point all the way through to the mitotic checkpoint and late mitosis. An example of an aurora kinase inhibitor includes, but is not limited to Binucleine 2, which is also known as methanimidamide, N-[1-(3-chloro-4-fluorophenyl)-4-cyano-1H-pyrazol-5-yl]-N,N-dimethyl-(9Cl).

The term “a Bruton's Tyrosine Kinase (BTK) inhibitor”, as used herein, relates to a compound which targets, decreases or inhibits human and murine B cell development. An example of a BTK inhibitor includes, but is not limited to, terreic acid.

The term “a calcineurin inhibitor”, as used herein, relates to a compound which targets, decreases or inhibits the T cell activation pathway. A target of a calcineurin inhibitor includes protein phosphatase 2B. Examples of a calcineurin inhibitor include, but are not limited to, Cypermethrin, which is also known as cyclopropanecarboxylic acid, 3-(2,2-dichloroethenyl)-2,2-dimethyl-,cyano(3-phenoxyphenyl)methyl ester (9Cl); Deltamethrin, which is also known as cyclopropanecarboxylic acid, 3-(2,2-dibromoethenyl)-2,2-dimethyl-(S)-cyano(3-phenoxyphenyl)methyl ester, (1R,3R)-(9Cl); Fenvalerate, which is also known as benzeneacetic acid, 4-chloro-α-(1-methylethyl)-,cyano(3-phenoxyphenyl)methyl ester (9Cl) and Tyrphostin 8.

The term “a CaM kinase II inhibitor”, as used herein, relates to a compound which targets, decreases or inhibits CaM Kinases. CaM Kinases constitute a family of structurally related enzymes that include phosphorylase kinase, myosin light chain kinase and CaM kinases I-IV. CaM Kinase II, one of the best-studied multifunctional enzymes, is found in high concentrations in neuronal synapses, and in some regions of the brain it may constitute up to 2% of the total protein content. Activation of CaM kinase II has been linked to memory and learning processes in the vertebrate nervous system. Targets of a CaM kinase II inhibitor include CaM kinase II. Examples of a CaM kinase II inhibitor include, but are not limited to, 5-Isoquinolinesulfonic acid, 4-[(2S)-2-[(5-isoquinolinylsulfonyl)methylamino]-3-oxo-3-(4-phenyl-1-piperazinyl)propyl]phenyl ester (9Cl); and benzenesulfonamide, N-[2-[[[3-(4-chlorophenyl)-2-propenyl]methyl]amino]methyl]phenyl]-N-(2-hydroxyethyl)-4-methoxy-9Cl).

The term “a CD45 tyrosine phosphatase inhibitor”, as used herein, relates to a compound which targets, decreases or inhibits dephosphorylating regulatory pTyr residues on Src-family protein-tyrosine kinases, which aids in the treatment of a variety of inflammatory and immune disorders. An example of a CD45 tyrosine phosphatase inhibitor includes, but is not limited to, phosphonic acid, [[2-(4-bromophenoxy)-5-nitrophenyl]hydroxymethyl]-(9Cl).

The term “a CDC25 phosphatase inhibitor”, as used herein, relates to compound which targets, decreases or inhibits overexpressed dephosphorylate cyclin-dependent kinases in tumors. An example of a CDC25 phosphatase inhibitor includes 1,4-naphthalenedione, 2,3-bis[(2-hydroxyethyl)thio]-(9Cl).

The term “a CHK kinase inhibitor”, as used herein, relates to a compound which targets, decreases or inhibits overexpression of the antiapoptotic protein Bcl-2. Targets of a CHK kinase inhibitor are CHK1 and/or CHK2. An example of a CHK kinase inhibitor includes, but is not limited to, debromohymenialdisine.

Examples of a “controlling agent for regulating genistein, olomucine and/or tyrphostins” includes, but are not limited to, daidzein, which is also known as 4H-1-benzopyran-4-one, 7-hydroxy-3-(4-hydroxyphenyl)-(9Cl), iso-olomoucine, and tyrphostin 1.

The term “cyclooxygenase inhibitor”, as used herein, includes, but is not limited to, e.g., Cox-2 inhibitors. The term “a COX-2 inhibitor”, as used herein, relates to a compound which targets, decreases or inhibits the enzyme cox-2 (cyclooxygenase-2). Examples of a COX-2 inhibitor include, but are not limited to, 1H-indole-3-acetamide, 1-(4-chlorobenzoyl)-5-methoxy-2-methyl-N-(2-phenylethyl)-(9Cl); 5-alkyl substituted 2-arylaminophenylacetic acid and derivatives, such as celecoxib (CELEBREX), rofecoxib (VIOXX), etoricoxib, valdecoxib; or a 5-alkyl-2-arylaminophenylacetic acid, e.g., 5-methyl-2-(2′-chloro-6′-fluoroanilino)phenyl acetic acid, lumiracoxib; and celecoxib.

The term “a cRAF kinase inhibitor”, as used herein, relates to a compound which targets, decreases or inhibits the up-regulation of E-selectin and vascular adhesion molecule-1 induced by TNF. Raf kinases play an important role as extracellular signal-regulating kinases in cell differentiation, proliferation and apoptosis. A target of a cRAF kinase inhibitor includes, but is not limited, to RAF1. Examples of a cRAF kinase inhibitor include, but are not limited to, 3-(3,5-dibromo-4-hydroxybenzylidene)-5-iodo-1,3-dihydroindol-2-one; and benzamide, 3-(dimethylamino)-N-[3-[(4-hydroxybenzoyl)amino]-4-methylphenyl]-(9Cl).

The term “a cyclin dependent kinase inhibitor”, as used herein, relates to a compound which targets, decreases or inhibits cyclin dependent kinase which play a role in the regulation of the mammalian cell cycle. Cell cycle progression is regulated by a series of sequential events that include the activation and subsequent inactivation of cyclin dependent kinases (Cdks) and cyclins. Cdks are a group of serine/threonine kinases that form active heterodimeric complexes by binding to their regulatory subunits, cyclins. Examples of targets of a cyclin dependent kinase inhibitor include, but are not limited to, CDK, AHR, CDK1, CDK2, CDK5, CDK4/6, GSK3beta and ERK. Examples of a cyclin dependent kinase inhibitor include, but are not limited to, N9-Isopropyl-Olomoucine; Olomoucine; Purvalanol B, which is also known as benzoic acid, 2-chloro-4-[[2-[[(1R)-1-(hydroxymethyl)-2-methylpropyl]amino]-9-(1-methylethyl)-9H-purin-6-yl]amino]-(9Cl); Roascovitine; Indirubin, which is also known as 2H-indol-2-one, 3-(1,3-dihydro-3-oxo-2H-indol-2-ylidene)-1,3-dihydro-(9Cl); Kenpaullone, which is also known as indolo[3,2-d][1]benzazepin-6(5H)-one, 9-bromo-7,12-dihydro-(9Cl); purvalanol A, which is also known as 1-Butanol, 2-[[6-[(3-chlorophenyl)amino]-9-(1-methylethyl)-9H-purin-2-yl]amino]-3-methyl-, (2R)-(9Cl); and indirubin-3′-monooxime.

The term “a cysteine protease inhibitor”, as used herein, relates to a compound which targets, decreases or inhibits cystein protease which plays a vital role in mammalian cellular turnover and apotosis. An example of a cystein protease inhibitor includes, but is not limited to, 4-morpholinecarboxamide, N-[(1S)-3-fluoro-2-oxo-1-(2-phenylethyl)propyl]amino]-2-oxo-1-(phenylmethyl)ethyl]-(9Cl).

The term “a DNA intercalator” as used herein, relates to a compound which binds to DNA and inhibits DNA, RNA and protein synthesis. Examples of a DNA intercalator include, but are not limited to, Plicamycin and Dactinomycin.

The term “a DNA strand breaker”, as used herein, relates to a compound which causes DNA strand scission and results in inhibition of DNA synthesis, inhibition of RNA and protein synthesis. An example of a DNA strand breaker includes, but is not limited to, Bleomycin.

The term “an E3 Ligase inhibitor”, as used herein, relates to a compound which targets, decreases or inhibits the E3 ligase which inhibits the transfer of ubiquitin chains to proteins, marking them for degradation in the proteasome. An example of a E3 ligase inhibitor includes, but is not limited to, N-((3,3,3-trifluoro-2-trifluoromethyl)propionyl)sulfanilamide.

The term “an endocrine hormone”, as used herein, relates to a compound which by acting mainly on the pituitary gland causes the suppression of hormones in males, the net effect is a reduction of testosterone to castration levels. In females, both ovarian estrogen and androgen synthesis are inhibited. An example of an endocrine hormone includes, but is not limited to, leuprolide and megestrol acetate.

The term “compounds targeting, decreasing or inhibiting the activity of the epidermal growth factor family”, as used herein, relates to a compound which icompounds targeting, decreasing or inhibiting the activity of the epidermal growth factor family of receptor tyrosine kinases (EGFR, ErbB2, ErbB3, ErbB4 as homo- or heteromers), such as compounds which target, decrease or inhibit the activity of the epidermal growth factor receptor family are especially compounds, proteins or antibodies which inhibit members of the EGF receptor tyrosine kinase family, e.g., EGF receptor, ErbB2, ErbB3 and ErbB4 or bind to EGF or EGF-related ligands, and are in particular those compounds, proteins or monoclonal antibodies generically and specifically disclosed in WO 97/02266, e.g., the compounds in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347, e.g., compound known as CP 358774, WO 96/33980, e.g., compound ZD 1839; and WO 95/03283, e.g., compound ZM105180, e.g., trastuzumab (HERCEPTIN®), cetuximab, Iressa, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541, erlotinib and gefitinib. Erlotinib can be administered in the form as it is marketed, e.g., TARCEVA, and gefitinib as IRESSA, human monoclonal antibodies against the epidermal growth factor receptor including ABX-EGFR. Targets of an EGFR kinase inhibitor include, but are not limited to, guanylyl cyclase (GC-C) and HER2. Other examples of an EGFR kinase inhibitor include, but are not limited to, Tyrphostin 23, Tyrphostin 25, Tyrphostin 47, Tyrphostin 51 and Tyrphostin AG 825. Targets of an EGFR tyrosine kinase inhibitor include EGFR, PTK and tubulin. Other examples of an EGFR tyrosine kinase inhibitor include, but are not limited to, 2-propenamide, 2-cyano-3-(3,4-dihydroxyphenyl)-N-phenyl-, (2E)-(9Cl); Tyrphostin Ag 1478; Lavendustin A; and 3-pyridineacetonitrile, α-[(3,5-dichlorophenyl)methylene]-, (αZ)-(9Cl). An example of an EGFR, PDGFR tyrosine kinase inhibitor includes, but is not limited to, Tyrphostin 46.

The term “a farnesyltransferase inhibitor”, as used herein, relates to a compound which targets, decreases or inhibits the Ras protein, which is commonly abnormally active in cancer. A target of a farnesyltransferase inhibitor includes, but is not limited to, RAS. Examples of a farnesyltransferase inhibitor include, but are not limited to, α-hydroxyfarnesylphosphonic acid, butanoic acid, 2-[[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-, 1-methylethyl ester, (2S)-(9Cl); and Manumycin A.

The term “a Flk-1 kinase inhibitor”, as used herein, relates to a compound which targets, decreases or inhibits Flk-1 tyrosine kinase activity. A target of a Flk-1 kinase inhibitor includes, but is not limited to, KDR. An example of a Flk-1 kinase inhibitor includes, but is not limited to, 2-propenamide, 2-cyano-3-[4-hydroxy-3,5-bis(1-methylethyl)phenyl]-N-(3-phenylpropyl)-, (2E)-(9Cl).

The term “a Glycogen synthase kinase-3 (GSK3) inhibitor”, as used herein, relates to a compound which targets, decreases or inhibits glycogen synthase kinase-3 (GSK3). Glycogen Synthase Kinase-3 (GSK-3; tau protein kinase I), a highly conserved, ubiquitously expressed serine/threonine protein kinase, is involved in the signal transduction cascades of multiple cellular processes, which is a protein kinase that has been shown to be involved in the regulation of a diverse array of cellular functions, including protein synthesis, cell proliferation, cell differentiation, microtubule assembly/disassembly and apoptosis. An example of a GSK3 inhibitor includes, but is not limited to, indirubin-3′-monooxime.

The term “a HDAC inhibitor”, as used herein, relates to a compound which inhibits the histone deacetylase and which possess anti-proliferative activity. This includes, but is not limited to, compounds disclosed in WO 02/22577, especially N-hydroxy-3-[4-[[(2-hydroxyethyl)[2-(1H-indol-3-yl)ethyl]-amino]methyl]phenyl]-2E-2-propenamide, and N-hydroxy-3-[4-[[[2-(2-methyl-1H-indol-3-yl)-ethyl]-amino]methyl]phenyl]-2E-2-propenamide and pharmaceutically acceptable salts thereof. It further includes suberoylanilide hydroxamic acid (SAHA); [4-(2-amino-phenylcarbamoyl)-benzyl]-carbamic acid pyridine-3-ylmethyl ester and derivatives thereof; butyric acid, pyroxamide, trichostatin A, Oxamflatin, apicidin, Depsipeptide; depudecin and trapoxin. Other examples include depudecin; HC Toxin, which is also known as cyclo[L-alanyl-D-alanyl-(αS,2S)-α-amino-α-oxooxiraneoctanoyl-D-prolyl] (9Cl); sodium phenylbutyrate, suberoyl bis-hydroxamic acid and Trichostatin A.

The term “HSP90 inhibitor”, as used herein, relates to a compound which targets, decreases or inhibits the intrinsic ATPase activity of HSP90; degrades, targets, decreases or inhibits the HSP90 client proteins via the ubiquitin proteosome pathway. Potential indirect targets of an HSP90 inhibitor include FLT3, BCR-ABL, CHK1, CYP3A5*3 and/or NQO1*2. Compounds targeting, decreasing or inhibiting the intrinsic ATPase activity of HSP90 are especially compounds, proteins or antibodies which inhibit the ATPase activity of HSP90, e.g., 17-allylamino, 17-demethoxygeldanamycin (17AAG), a geldanamycin derivative; other geldanamycin-related compounds; radicicol and HDAC inhibitors. Other examples of an HSP90 inhibitor include geldanamycin, 17-demethoxy-17-(2-propenylamino)-(9Cl) and geldanamycin.

The term “a I-kappa B-alpha kinase inhibitor (IKK)”, as used herein, relates to a compound which targets, decreases or inhibits NF-kappaB. An example of an IKK inhibitor includes, but is not limited to, 2-propanenitrile, 3-[(4-methylphenyl)sulfonyl]-, (2E)-(9Cl).

The term “an insulin receptor tyrosine kinase inhibitor”, as used herein, relates to a compound which modulates the activities of phosphatidylinositol 3-kinase, microtubule-associated protein and S6 kinases. An example of an insulin receptor tyrosine kinase inhibitor includes, but is not limited to, hydroxyl-2-naphthalenylmethylphosphonic acid.

The term “a c-Jun N-terminal kinase (JNK) kinase inhibitor”, as used herein, relates to a compound which targets, decreases or inhibits Jun N-terminal kinase. Jun N-terminal kinase (JNK), a serine-directed protein kinase, is involved in the phosphorylation and activation of c-Jun and ATF2 and plays a significant role in metabolism, growth, cell differentiation and apoptosis. A target for a JNK kinase inhibitor includes, but is not limited to, DNMT. Examples of a JNK kinase inhibitor include, but are not limited to, pyrazoleanthrone and/or epigallocatechin gallate.

The term “a microtubule binding agent”, as used herein, refers to a compound which acts by disrupting the microtubular network that is essential for mitotic and interphase cellular function. Examples of a microtubule binding agent include, but are not limited to, vinblastine sulfate; vincristine sulfate; vindesine; vinorelbine; docetaxel; paclitaxel; vinorelbine; discodermolides; cochicine and epothilonesand derivatives thereof, e.g., epothilone B or a derivative thereof. Paclitaxel is marketed as TAXOL; docetaxel as TAXOTERE; vinblastine sulfate as VINBLASTIN R.P; and vincristine sulfate as FARMISTIN. Also included are the generic forms of paclitaxel as well as various dosage forms of paclitaxel. Generic forms of paclitaxel include, but are not limited to, betaxolol hydrochloride. Various dosage forms of paclitaxel include, but are not limited to, albumin nanoparticle paclitaxel marketed as ABRAXANE; ONXOL, CYTOTAX. Discodermolide can be obtained, e.g., as disclosed in U.S. Pat. No. 5,010,099. Also included are epotholine derivatives which are disclosed in U.S. Pat. No. 6,194,181, WO 98/10121, WO 98/25929, WO 98/08849, WO 99/43653, WO 98/22461 and WO 00/31247. Especially preferred are Epotholine A and/or B.

The term “a mitogen-activated protein (MAP) kinase inhibitor”, as used herein, relates to a compound which targets, decreases or inhibits mitogen-activated protein. The MAP kinases are a group of protein serine/threonine kinases that are activated in response to a variety of extracellular stimuli and mediate signal transduction from the cell surface to the nucleus. They regulate several physiological and pathological cellular phenomena, including inflammation, apoptotic cell death, oncogenic transformation, tumor cell invasion and metastasis. An example of a MAP kinase inhibitor includes, but is not limited to, benzenesulfonamide, N-[2-[[[3-(4-chlorophenyl)-2-propenyl]methyl]amino]methyl]phenyl]-N-(2-hydroxyethyl)-4-methoxy-(9Cl).

The term “a MDM2 inhibitor”, as used herein, relates to a compound which targets, decreases or inhibits the interaction of MDM2 and the p53 tumor suppressor. An example of a MDM2 inhibitor includes, but is not limited to, trans-4-iodo, 4′-boranyl-chalcone.

The term “a MEK inhibitor”, as used herein, relates to a compound which targets, decreases or inhibits the kinase activity of MAP kinase, MEK. A target of a MEK inhibitor includes, but is not limited to, ERK. An indirect target of a MEK inhibitor includes, but is not limited to, cyclin D1. An example of a MEK inhibitor includes, but is not limited to, butanedinitrile, bis[amino[2-aminophenyl)thio]methylene]-(9Cl).

The term “a MMP inhibitor”, as used herein, relates to a compound which targets, decreases or inhibits a class of protease enzyme that selectively catalyze the hydrolysis of polypeptide bonds including the enzymes MMP-2 and MMP-9 that are involved in promoting the loss of tissue structure around tumours and facilitating tumour growth, angiogenesis and metastasis. A target of a MMP inhibitor includes, but is not limited to, polypeptide deformylase. Example of a MMP inhibitor include, but are not limited to, actinonin, which is also known as butanediamide, N4-hydroxy-N1-[(1S)-1-[[(2S)-2-(hydroxymethyl)-1-pyrrolidinyl]carbonyl]-2-methylpropyl]-2-pentyl-, (2R)-(9Cl); epigallocatechin gallate; collagen peptidomimetic and non-peptidomimetic inhibitors; tetracycline derivatives, e.g., hydroxamate peptidomimetic inhibitor batimastat; and its orally-bioavailable analogue marimastat, prinomastat, metastat, Neovastat, Tanomastat, TAA211, MMI270B or AAJ996.

The term “a NGFR tyrosine-kinase-inhibitor”, as used herein, relates to a compound which targets, decreases or inhibits nerve growth factor dependent p140^{c-trk} tyrosine phosphorylation. Targets of a NGFR tyrosine-kinase-inhibitor include, but are not limited to, HER2, FLK1, FAK, TrkA and/or TrkC. An indirect target inhibits expression of RAF1. An example of a NGFR tyrosine-kinase-inhibitor includes, but is not limited to, Tyrphostin AG 879.

The term “a p38 MAP kinase inhibitor”, as used herein, relates to a compound which targets, decreases or inhibits p38-MAPK, which is a MAPK family member. A MAPK family member is a serine/threonine kinase activated by phosphorylation of tyrosine and threonine residues. This kinase is phosphorylated and activated by many cellular stresses and inflammatory stimuli, thought to be involved in the regulation of important cellular responses, such as apoptosis and inflammatory reactions. An example of a p38 MAP kinase inhibitor includes, but is not limited to, phenol, 4-[4-(4-fluorophenyl)-5-(4-pyridinyl)-1H-imidazol-2-yl]-(9Cl). An example of a SAPK2/p38 kinase inhibitor includes, but is not limited to, benzamide, 3-(dimethylamino)-N-[3-[(4-hydroxybenzoyl)amino]-4-methylphenyl]-(9Cl).

The term “a p56 tyrosine kinase inhibitor”, as used herein, relates to a compound which targets, decreases or inhibits p56 tyrosine kinase, which is an enzyme that is a lymphoid-specific src family tyrosine kinase critical for T-cell development and activation. A target of a p56 tyrosine kinase inhibitor includes, but is not limited to, Lck. Lck is associated with the cytoplasmic domains of CD4, CD8 and the beta-chain of the IL-2 receptor, and is thought to be involved in the earliest steps of TCR-mediated T-cell activation. Examples of a p56 tyrosine kinase inhibitor include, but are not limited to, damnacanthal, which is also known as 2-anthracenecarboxaldehyde, 9,10-dihydro-3-hydroxy-1 methoxy-9, I O-dioxo-(9Cl) and/or Tyrphostin 46.

The term “a PDGFR tyrosine kinase inhibitor”, as used herein, relates to compounds targeting, decreasing or inhibiting the activity of the C-kit receptor tyrosine kinases (part of the PDGFR family), such as compounds which target, decrease or inhibit the activity of the c-Kit receptor tyrosine kinase family, especially compounds which inhibit the c-Kit receptor, PDGF plays a central role in regulating cell proliferation, chemotaxis, and survival in normal cells, as well as in various disease states such as cancer, atherosclerosis and fibrotic disease. The PDGF family is composed of dimeric isoforms (PDGF-AA, PDGF-BB, PDGF-AB, PDGF-CC and PDGF-DD), which exert their cellular effects by differentially binding to two receptor tyrosine kinases. PDGFR-α and PDGFR-β have molecular masses of ˜170 and 180 kDa, respectively. Examples of targets of a PDGFR tyrosine kinase inhibitor includes, but are not limited to, PDGFR, FLT3 and/or c-KIT. Examples of a PDGFR tyrosine kinase inhibitor include, but are not limited to, Tyrphostin AG 1296; Tyrphostin 9; 1,3-butadiene-1,1,3-tricarbonitrile,2-amino-4-(1H-indol-5-yl)-(9Cl); imatinib and IRESSA.

The term “a phosphatidylinositol 3-kinase inhibitor”, as used herein, relates to a compound which targets, decreases or inhibits PI 3-kinase. PI 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation. An example of a target of a phosphatidylinositol 3-kinase inhibitor includes, but is not limited to, Pi3K. Examples of a phosphatidylinositol 3-kinase inhibitor include, but are not limited to, Wortmannin, which is also known as 3H-furo[4,3,2-de]indeno[4,5-h]-2-benzopyran-3,6,9-trione, 11-(acetyloxy)-1,6b,7,8,9a,10,11,11 b-octahydro-1-(methoxymethyl)-9a,11b-dimethyl-, (1S,6bR,9aS,11R,11bR)-(9Cl); 8-phenyl-2-(morpholin-4-yl)-chromen-4-one; and/or quercetin dihydrate.

The term “a phosphatase inhibitor”, as used herein, relates to a compound which targets, decreases or inhibits phosphatase. Phosphatases remove the phosphoryl group and restore the protein to its original dephosphorylated state. Hence, the phosphorylation-dephosphorylation cycle can be regarded as a molecular “on-off” switch. Examples of a phosphatase inhibitor include, but are not limited to, cantharidic acid; cantharidin; and L-leucinamide, N-[4-(2-carboxyethenyl)benzoyl]glycyl-L-α-glutamyl-, (E)-(9Cl).

The term “a platinum agent”, as used herein, relates to a compound which contains Platinum and inhibit DNA synthesis by forming interstrand and intrastrand cross-linking of DNA molecules. Examples of a platinum agent include, but are not limited to, carboplatin; cisplatin; oxaliplatin; cisplatinum; satraplatin and platinum agents, such as ZD0473. Carboplatin can be administered, e.g., in the form as it is marketed, e.g., CARBOPLAT; and oxaliplatin as ELOXATIN.

The term “a protein phosphatase inhibitor”, as used herein, relate to a compound which targets, decreases or inhibits protein phosphatase. The term “a PP1 or PP2 inhibitor”, as used herein, relates to a compound which targets, decreases or inhibits Ser/Thr protein phosphatases. Type I phosphatases, which include PP1, can be inhibited by two heat-stable proteins known as Inhibitor-1 (I-1) and Inhibitor-2 (I-2). They preferentially dephosphorylate the α-subunit of phosphorylase kinase. Type II phosphatases are subdivided into spontaneously active (PP2A), CA²⁺-dependent (PP2B) and Mg²⁺-dependent (PP2C) classes of phosphatases. Examples of a PP1 and PP2A inhibitor include, but are not limited to, cantharidic acid and/or cantharidin. The term “tyrosine phosphatase inhibitor”, as used herein, relates to a compounds which targets, decreases or inhibits tyrosine phosphatase. Protein tyrosine phosphatases (PTPs) are relatively recent additions to the phosphatase family. They remove phosphate groups from phosphorylated tyrosine residues of proteins. PTPs display diverse structural features and play important roles in the regulation of cell proliferation, differentiation, cell adhesion and motility and cytoskeletal function. Examples of targets of a tyrosine phosphatase inhibitor include, but are not limited to, alkaline phosphatase (ALP), heparanase, PTPase and/or prostatic acid phosphatase. Examples of a tyrosine phosphatase inhibitor include, but are not limited to, L-P-bromotetramisole oxalate; 2(5M-furanone, 4-hydroxy-5-(hydroxymethyl)-3-(1-oxohexadecyl)-, (5R)-(9Cl) and benzylphosphonic acid.

The term “a PKC inhibitor”, as used herein, relates to a compound which targets, decreases or inhibits protein kinase C(PKC), as well as its isozymes. PKC, a ubiquitous, phospholipid-dependent enzyme, is involved in signal transduction associated with cell proliferation, differentiation and apoptosis. Examples of a target of a PKC inhibitor include, but are not limited to, MAPK and/or NF-kappaB. Examples of a PKC inhibitor include, but are not limited to, 1H-pyrrolo-2,5-dione,3-[1-[3-(dimethylamino)propyl]-1H-indol-3-yl]-4-(1H-indol-3-yl)-(9Cl); bisindolylmaleimide IX; sphingosine, which is known as 4-octadecene-1,3-diol, 2-amino-, (2S,3R,4E)-(9Cl); staurosporine, which is known as 9,13-epoxy-1H,9H-diindolo[1,2,3-gh:3′,2′,1′-lm]pyrrolo[3,4-j][1,7]benzodiazonin-1-one, 2,3,10,11,12,13-hexahydro-10-methoxy-9-methyl-11-(methylamino)-, (9S,10R,11R,13R)-(9Cl); tyrphostin 51; and hypericin, which is also known as phenanthro[1,10,9,8-opqra]perylene-7,14-dione, 1,3,4,6,8,13-hexahydroxy-10,11-dimethyl-, stereoisomer (6Cl, 7Cl, 8Cl,9Cl).

The term “a PKC delta kinase inhibitor”, as used herein, relates to a compound which targets, decreases or inhibits the delta isozymes of PKC. The delta isozyme is a conventional PKC isozymes and is Ca²⁺-dependent. An example of a PKC delta kinase inhibitor includes, but is not limited to, rottlerin, which is also known as 2-propen-1-one, 1-[6-[(3-acetyl-2,4,6-trihydroxy-5-methylphenyl)methyl]-5,7-dihydroxy-2,2-dimethyl-2H-1-benzopyran-8-yl]-3-phenyl-, (2E)-(9Cl).

The term “a polyamine synthesis inhibitor”, as used herein, relates to a compound which targets, decreases or inhibits polyamines spermidine. The polyamines spermidine and spermine are of vital importance for cell proliferation, although their precise mechanism of action is unclear. Tumor cells have an altered polyamine homeostasis reflected by increased activity of biosynthetic enzymes and elevated polyamine pools. Examples of a polyamine synthesis inhibitor include, but are not limited to, DMFO, which is also known as (−)-2-difluoromethylornithin; N1, N12-diethylspermine 4HCl.

The term “a proteosome inhibitor”, as used herein, relates to a compound which targets, decreases or inhibits proteasome. Examples of targets of a proteosome inhibitor include, but are not limited to, O(2)(−)-generating NADPH oxidase, NF-kappaB, and/or farnesyltransferase, geranylgeranyltransferase I. Examples of a proteosome inhibitor include, but are not limited to, aclacinomycin A; gliotoxin; PS-341; MLN 341; bortezomib; or Velcade.

The term “a PTP1B inhibitor”, as used herein, relates to a compound which targets, decreases or inhibits PTP1B, a protein tyrosine kinase inhibitor. An example of a PTP1B inhibitor includes, but is not limited to, L-leucinamide, N-[4-(2-carboxyethenyl)benzoyl]glycyl-L-α-glutamyl-, (E)-(9Cl).

The term “a protein tyrosine kinase inhibitor”, as used herein, relates to a compound which targets, decreases or inhibits PTKs. PTKs play a key role in the regulation of cell proliferation, differentiation, metabolism, migration and survival. They are classified as receptor PTKs and non-receptor PTKs. Receptor PTKs contain a single polypeptide chain with a transmembrane segment. The extracellular end of this segment contains a high affinity ligand-binding domain, while the cytoplasmic end comprises the catalytic core and the regulatory sequences. Examples of targets of a tyrosine kinase inhibitor include, but are not limited to, ERK1, ERK2, Bruton's tyrosine kinase (Btk), JAK2, ERK 1/2, PDGFR and/or FLT3. Examples of indirect targets include, but are not limited to, TNFalpha, NO, PGE2, IRAK, iNOS, ICAM-1 and/or E-selectin. Examples of a tyrosine kinase inhibitor include, but are not limited to, Tyrphostin AG 126; Tyrphostin Ag 1288; Tyrphostin Ag 1295; Geldanamycin; and Genistein.

Non-receptor tyrosine kinases include members of the Src, Tec, JAK, Fes, Abl, FAK, Csk and Syk families. They are located in the cytoplasm as well as in the nucleus. They exhibit distinct kinase regulation, substrate phosphorylation and function. Deregulation of these kinases has also been linked to several human diseases.

The term “a SRC family tyrosine kinase inhibitor”, as used herein, relates to a compound which targets, decreases or inhibits SRC. Examples of a SRC family tyrosine kinase inhibitor include, but are not limited to, PP1, which is also known as 1H-pyrazolo[3,4-d]pyrimidin-4-amine, 1-(1,1-dimethylethyl)-3-(1-naphthalenyl)-(9Cl); and PP2, which is also known as 1H-pyrazolo[3,4-d]pyrimidin-4-amine, 3-(4-chlorophenyl)-1-(1,1-dimethylethyl)-(9Cl).

The term “a Syk tyrosine kinase inhibitor”, as used herein, relates to a compound which targets, decreases or inhibits Syk. Examples of targets for a Syk tyrosine kinase inhibitor include, but are not limited to, Syk, STAT3 and/or STAT5. An example of a Syk tyrosine kinase inhibitor includes, but is not limited to, piceatannol, which is also known as 1,2-benzenediol, 4-[(1E)-2-(3,5-dihydroxyphenyl)ethenyl]-(9Cl).

The term “a Janus (JAK-2 and/or JAK-3) tyrosine kinase inhibitor”, as used herein, relates to a compound which targets, decreases or inhibits janus tyrosine kinase. Janus tyrosine kinase inhibitor are shown anti-leukemic agents with anti-thrombotic, anti-allergic and immunosuppressive properties. Targets of a JAK-2 and/or JAK-3 tyrosine kinase inhibitor include, but are not limited to, JAK2, JAK3, STAT3. An indirect target of an JAK-2 and/or JAK-3 tyrosine kinase inhibitor includes, but is not limited to CDK2. Examples of a JAK-2 and/or JAK-3 tyrosine kinase inhibitor include, but are not limited to, tyrphostin AG 490; and 2-naphthyl vinyl ketone.

The term “a retinoid”, as used herein, refers to compounds that target, decrease or inhibit retinoid dependent receptors. Examples include, but are not limited to Isotretinoin and Tretinoin.

The term “a RNA polymerase II elongation inhibitor”, as used herein, relates to a compound which targets, decreases or inhibits insulin-stimulated nuclear and cytosolic p70S6 kinase in CHO cells; targets, decreases or inhibits RNA polymerase II transcription, which may be dependent on casein kinase II; and targets, decreases or inhibits germinal vesicle breakdown in bovine oocytes. An example of a RNA polymerase II elongation inhibitor includes, but is not limited to, 5,6-dichloro-1-beta-D-ribofuranosylbenzimidazole.

The term “a serine/threonine kinase inhibitor”, as used herein, relates to a compound which inhibits serine/threonine kinases. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-kappaB, elF2alpha, COX2, RANTES, IL8,CYP2A5, IGF-1, CYP2B1, CYP2B2, CYP2H1, ALAS-1, HIF-1, erythropoietin and/or CYP1A1. An example of a serine/theronin kinase inhibitor includes, but is not limited to, 2-aminopurine, also known as 1H-purin-2-amine(9Cl).

The term “a sterol biosynthesis inhibitor”, as used herein, relates to a compound which inhibits the biosynthesis of sterols, such as cholesterol Examples of targets for a sterol biosynthesis inhibitor include, but are not limited to, squalene epoxidase and CYP2D6. An example of a sterol biosynthesis inhibitor includes, but is not limited to, terbinadine.

The term “a topoisomerase inhibitor”, includes a topoisomerase I inhibitor and a topoisomerase II inhibitor. Examples of a topoisomerase I inhibitor include, but are not limited to, topotecan, gimatecan, irinotecan, camptothecian and its analogues, 9-nitrocamptothecin and the macromolecular camptothecin conjugate PNU-166148 (compound Al in WO 99/17804); 10-hydroxycamptothecin acetate salt; etoposide; idarubicin hydrochloride; irinotecan hydrochloride; teniposide; topotecan hydrochloride; doxorubicin; epirubicin hydrochloride; mitoxantrone hydrochloride; and daunorubicin hydrochloride. Irinotecan can be administered, e.g., in the form as it is marketed, e.g., under the trademark CAMPTOSAR. Topotecan can be administered, e.g., in the form as it is marketed, e.g., under the trademark HYCAMTIN. The term “topoisomerase II inhibitor”, as used herein, includes, but is not limited to, the anthracyclines, such as doxorubicin, including liposomal formulation, e.g., CAELYX, daunorubicin, including liposomal formulation, e.g., DAUNOSOME, epirubicin, idarubicin and nemorubicin; the anthraquinones mitoxantrone and losoxantrone; and the podophillotoxines etoposide and teniposide. Etoposide is marketed as ETOPOPHOS; teniposide as VM 26-BRISTOL; doxorubicin as ADRIBLASTIN or ADRIAMYCIN; epirubicin as FARMORUBICIN; idarubicin as ZAVEDOS; and mitoxantrone as NOVANTRON.

The term “VEGFR tyrosine kinase inhibitor”, as used herein, relates to a compound which targets, decreases and/or inhibits the known angiogenic growth factors and cytokines implicated in the modulation of normal and pathological angiogenesis. The VEGF family (VEGF-A, VEGF-B, VEGF-C, VEGF-D) and their corresponding receptor tyrosine kinases [VEGFR-1 (Flt-1), VEGFR-2 (Flk-1, KDR), and VEGFR-3 (Flt-4)] play a paramount and indispensable role in regulating the multiple facets of the angiogenic and lymphangiogenic processes. An example of a VEGFR tyrosine kinase inhibitor includes, but is not limited to, 3-(4-dimethylaminobenzylidenyl)-2-indolinone.

In each case where citations of patent applications or scientific publications are given, in particular with regard to the respective compound claims and the final products of the working examples therein, the subject matter of the final products, the pharmaceutical preparations and the claims is hereby incorporated into the present application by reference to these publications. Comprised are likewise the corresponding stereoisomers, as well as the corresponding crystal modifications, e.g., solvates and polymorphs, which are disclosed therein. The compounds used as active ingredients in the combinations disclosed herein can be prepared and administered as described in the cited documents, respectively.

The structure of the active agents identified by code numbers, generic or trade names may be taken from the actual edition of the standard compendium “The Merck Index” or from databases, e.g., Patents International, e.g., IMS World Publications, or the publications mentioned above and below. The corresponding content thereof is hereby incorporated by reference.

It will be understood that references to the components (a) and (b) are meant to also include the pharmaceutically acceptable salts of any of the active substances. If active substances comprised by components (a) and/or (b) have, e.g., at least one basic center, they can form acid addition salts. Corresponding acid addition salts can also be formed having, if desired, an additionally present basic center. Active substances having an acid group, e.g., COOH, can form salts with bases. The active substances comprised in components (a) and/or (b) or a pharmaceutically acceptable salts thereof may also be used in form of a hydrate or include other solvents used for crystallization. 7H-pyrrolo[2,3-d]pyrimidine derivative, {6-[4-(4-ethyl-piperazin-1-ylmethyl)-phenyl]-7H-pyrrolo[2,3-d]pyrimidin-4-yl]-((R)-1-phenyl-ethyl)-amine is the most preferred combination partner (a).

### III. The Combinations

The present invention relates to a combination of:

(a) an Erb-B and VEGF receptor inhibitor compound; and

(b) an pharmaceutically active agent.

In preferred embodiment, the present invention provides a combination comprising:
(a) an Erb-B and VEGF receptor inhibitor compound; and
(b) one or more pharmaceutically active agents selected from the group consisting of an inhibitor of apoptosis proteins, a steroid, an antimetabolite; a MEK inhibitor; a PKC inhibitor, a protein tyrosine kinase inhibitor and topoisomerase inhibitor.

In another preferred embodiment, the present invention provides a combination comprising:
(a) an Erb-B and VEGF receptor inhibitor compound; and
(b) one or more pharmaceutically active agents selected from the group consisting of N-[1-cyclohexyl-2-oxo-2-(6-phenethyl-octahydro-pyrrolo[2,3-c]pyridin-1-yl-ethyl]-2-methylamino-propionamide, floxuridine, prednisone, cytarabine; cladribine, butanedinitrile, staurosporine; teniposide; mitoxantrone hydrochloride; etoposide.

In preferred embodiment, the present invention provides a combination comprising:
(a) an Erb-B and VEGF receptor inhibitor compound of formula I; and
(b) one or more pharmaceutically active agents selected from the group consisting of an inhibitor of apoptosis proteins, a steroid, an antimetabolite; a MEK inhibitor; a PKC inhibitor; a protein tyrosine kinase inhibitor and topoisomerase inhibitor.

In another preferred embodiment, the present invention provides a combination comprising:
(a) an Erb-B and VEGF receptor inhibitor compound of formula (I); and
(b) one or more pharmaceutically active agents selected from the group consisting of N-[1-cyclohexyl-2-oxo-2-(6-phenethyl-octahydro-pyrrolo[2,3-c]pyridin-1-yl-ethyl]-2-methylamino-propionamide, floxuridine, prednisone, cytarabine; cladribine, butanedinitrile, staurosporine; teniposide; mitoxantrone hydrochloride; etoposide.

In preferred embodiment, the present invention provides a combination comprising:
(a) 7H-pyrrolo[2,3-d]pyrimidine derivative, {6-[4-(4-ethyl-piperazin-1-ylmethyl)-phenyl]-7H-pyrrolo[2,3-d]pyrimidin-4-yl]-((R)-1-phenyl-ethyl)-amine; and
(b) one or more pharmaceutically active agents selected from the group consisting of an inhibitor of apoptosis proteins, a steroid, an antimetabolite; a MEK inhibitor; a PKC inhibitor; a protein tyrosine kinase inhibitor and topoisomerase inhibitor.

In another preferred embodiment, the present invention provides a combination comprising:
(a) 7H-pyrrolo[2,3-d]pyrimidine derivative, {6-[4-(4-ethyl-piperazin-1-ylmethyl)-phenyl]-7H-pyrrolo[2,3-d]pyrimidin-4-yl]-((R)-1-phenyl-ethyl)-amine; and
(b) one or more pharmaceutically active agents selected from the group consisting of N-[1-cyclohexyl-2-oxo-2-(6-phenethyl-octahydro-pyrrolo[2,3-c]pyridin-1-yl-ethyl]-2-methylamino-propionamide, floxuridine, prednisone, Cytarabine; Cladribine, butanedinitrile, staurosporine; teniposide; mitoxantrone hydrochloride; etoposide.

Any of the combination of components (a) and (b), the method of treating a warm-blooded animal comprising administering these two components, a pharmaceutical composition comprising these two components for simultaneous, separate or sequential use, the use of the combination for the delay of progression or the treatment of a proliferative disease or for the manufacture of a pharmaceutical preparation for these purposes or a commercial product comprising such a combination of components (a) and (b), all as mentioned or defined above, will be referred to subsequently also as COMBINATION OF THE INVENTION (so that this term refers to each of these embodiments which thus can replace this term where appropriate).

### IV. Administration

Simultaneous administration may, e.g., take place in the form of one fixed combination with two or more active ingredients, or by simultaneously administering two or more active ingredients that are formulated independently. Sequential use (administration) preferably means administration of one (or more) components of a combination at one time point, other components at a different time point, that is, in a chronically staggered manner, preferably such that the combination shows more efficiency than the single compounds administered independently (especially showing synergism). Separate use (administration) preferably means administration of the components of the combination independently of each other at different time points, preferably meaning that the components (a) and (b) are administered such that no overlap of measurable blood levels of both compounds are present in an overlapping manner (at the same time).

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

The term “delay of progression”, as used herein, means administration of the combination to patients being in a pre-stage or in an early phase, of the first manifestation or a relapse of the disease to be treated, in which patients, e.g., a pre-form of the corresponding disease is diagnosed or which patients are in a condition, e.g., during a medical treatment or a condition resulting from an accident, under which it is likely that a corresponding disease will develop.

“Jointly therapeutically active” or “joint therapeutic effect” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect). Whether this is the case, can inter alia be determined by following the blood levels, showing that both compounds are present in the blood of the human to be treated at least during certain time intervals.

“Pharmaceutically effective” preferably relates to an amount that is therapeutically or in a broader sense also prophylactically effective against the progression of a proliferative disease.

### V. Commercial Package

The term “a commercial package” or “a product”, as used herein defines especially a “kit of parts” in the sense that the components (a) and (b) as defined above can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) (as can be determined according to standard methods. The ratio of the total amounts of the combination partner (a) to the combination partner (b) to be administered in the combined preparation can be varied, e.g., in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components) (a) and (b), and very preferably a strong synergism of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal anti-tumor effect) or the like.

The COMBINATION OF THE INVENTION can also be applied in combination with other treatments, e.g., surgical intervention, hyperthermia and/or irradiation therapy.

### VI. Pharmaceutical Compositions & Preparations

The pharmaceutical compositions according to the present invention can be prepared by conventional means and are those suitable for enteral, such as oral or rectal, and parenteral administration to mammals including man, comprising a therapeutically effective amount of a VEGF inhibitor and at least one pharmaceutically active agent alone or in combination with one or more pharmaceutically acceptable carriers, especially those suitable for enteral or parenteral application.

The pharmaceutical compositions comprise from about 0.00002% to about 100%, especially, e.g., in the case of infusion dilutions that are ready for use) of 0.0001-0.02%, or, e.g., in case of injection or infusion concentrates or especially parenteral formulations, from about 0.1% to about 95%, preferably from about 1% to about 90%, more preferably from about 20% to about 60%—DISCUSS active ingredient (weight by weight, in each case). Pharmaceutical compositions according to the invention may be, e.g., in unit dose form, such as in the form of ampoules, vials, dragees, tablets, infusion bags or capsules.

The effective dosage of each of the combination partners employed in a formulation of the present invention may vary depending on the particular compound or pharmaceutical compositions employed, the mode of administration, the condition being treated and the severity of the condition being treated. A physician, clinician or veterinarian of ordinary skill can readily determine the effective amount of each of the active ingredients necessary to prevent, treat or inhibit the progress of the condition.

Pharmaceutical preparations for the combination therapy for enteral or parenteral administration are, e.g., those in unit dosage forms, such as sugar-coated tablets, capsules or suppositories, and furthermore ampoules. If not indicated otherwise, these formulations are prepared by conventional means, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units. One of skill in the art has the ability to determine appropriate pharmaceutically effective amounts of the combination components. Preferably, the compounds or the pharmaceutically acceptable salts thereof, are administered as an oral pharmaceutical formulation in the form of a tablet, capsule or syrup; or as parenteral injections if appropriate.

In preparing compositions for oral administration, any pharmaceutically acceptable media may be employed, such as water, glycols, oils, alcohols, flavoring agents, preservatives, coloring agents. Pharmaceutically acceptable carriers include starches, sugars, microcrystalline celluloses, diluents, granulating agents, lubricants, binders, disintegrating agents.

Solutions of the active ingredient, and also suspensions, and especially isotonic aqueous solutions or suspensions, are useful for parenteral administration of the active ingredient, it being possible, e.g., in the case of lyophilized compositions that comprise the active ingredient alone or together with a pharmaceutically acceptable carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers, and are prepared in a manner known per se, e.g., by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semi-synthetic oils customary for injection purposes.

The isotonic agent may be selected from any of those known in the art, e.g., mannitol, dextrose, glucose and sodium chloride. The infusion formulation may be diluted with the aqueous medium. The amount of aqueous medium employed as a diluent is chosen according to the desired concentration of active ingredient in the infusion solution. Infusion solutions may contain other excipients commonly employed in formulations to be administered intravenously, such as antioxidants.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g., in order to cope with the needs of a patient sub-population to be treated or the needs of the single patient based on the severity of any side effects that the patient experiences.

The present invention especially relates to a combined preparation which comprises:

(a) one or more unit dosage forms of an Erb-B and VEGF receptor inhibitor; and

(b) one or more unit dosage forms of an pharmaceutically active agent.

### VII. The Diseases to be Treated

The compositions of the present invention are useful for treating proliferative diseases or diseases that are associated with or triggered by persistent angiogenesis.

A proliferative disease is mainly a tumor disease (or cancer) (and/or any metastases). The inventive compositions are particularly useful for treating a tumor which is a breast cancer, genitourinary cancer, lung cancer, gastrointestinal cancer, epidermoid cancer, melanoma, glioma, ovarian cancer, pancreas cancer, neuroblastoma, head and/or neck cancer or bladder cancer, or in a broader sense renal, brain or gastric cancer.

In particular, the inventive compositions are particularly useful for treating:
(i) a breast tumor; a lung tumor, e.g., a small cell or non-small cell lung tumor; melanoma; or
(ii) a proliferative disease that is refractory to the treatment with other chemotherapeutics; or
(iii) a tumor that is refractory to treatment with other chemotherapeutics due to multidrug resistance.

Where a tumor, a tumor disease, a carcinoma or a cancer are mentioned, also metastasis in the original organ or tissue and/or in any other location are implied alternatively or in addition, whatever the location of the tumor and/or metastasis.

The compositions are selectively toxic or more toxic to rapidly proliferating cells than to normal cells, particularly in human cancer cells, e.g., cancerous tumors, the compound has significant anti-proliferative effects and promotes differentiation, e.g., cell cycle arrest and apoptosis.

The invention is illustrated by the following Examples.

The following Examples illustrate the combinations with 7H-pyrrolo[2,3-d]pyrimidine derivative, {6-[4-(4-ethyl-piperazin-1-ylmethyl)-phenyl]-7H-pyrrolo[2,3-d]pyrimidin-4-yl]-((R)-1-phenyl-ethyl)-amine that show a syngeristic effect. All combinations were tested in three (3) distinct cell lines as part of this collaboration: A549, a model of non-small cell lung carcinoma; SKOV-3, a model of ovarian cancer; and SKMEL-28, a model of malignant melanoma.

One example is the synergistic effect observed between 7H-pyrrolo[2,3-d]pyrimidine derivative, {6-[4-(4-ethyl-piperazin-1-ylmethyl)-phenyl]-7H-pyrrolo[2,3-d]pyrimidin-4-yl]-((R)-1-phenyl-ethyl)-amine and N-[1-cyclohexyl-2-oxo-2-(6-phenethyl-octahydro-pyrrolo[2,3-c]pyridin-1-yl-ethyl]-2-methylamino-propionamide in SKOV-3 cells.

Another example is the synergistic effect observed between 7H-pyrrolo[2,3-d]pyrimidine derivative, {6-[4-(4-ethyl-piperazin-1-ylmethyl)-phenyl]-7H-pyrrolo[2,3-d]pyrimidin-4-yl]-((R)-1-phenyl-ethyl)-amine and the antimetabolite cladribine in A549 cells. A significant increase in the potency of 7H-pyrrolo[2,3-d]pyrimidine derivative, {6-[4-(4-ethyl-piperazin-1-ylmethyl)-phenyl]-7H-pyrrolo[2,3-d]pyrimidin-4-yl]-((R)-1-phenyl-ethyl)-amine was observed in the presence of increasing concentrations of cladribine.

All combinations were prepared in the same manner for testing.

### Assay Conditions and Protocols

### Day 1: Cell Preparation

Cells were cultured in T-175 flasks in complete medium (RPMI-1640, 10% FBS, 1% Penn/Strep) at 37° C. and 5% CO₂. Cells were removed from the flask by brief treatment with 0.25% trypsin. Trypsin was inactivated with media and cell count was adjusted appropriately. Cells were then seeded into 384-well microtiter plates (35 μL) at 1500 (A549) or 3,000 (SKOV-3, SKMEL-28) cells/well using a multi-drop 16-24 hours prior to compound addition for general screening. Seeded plates were incubated (37° C./5% CO₂) overnight to allow recovery and re-attachment.

### Day 2: Compound Addition

Dilution plates were prepared with 100 μL per well of complete medium non-cell culture treated polypropylene 384-well plates. Compounds were added to dilution plates using the Mini-Trak (1 μL addition) for a 1:101 dilution followed by mixing. For single agent dose response curves, a 5 μL aliquot from a dilution plate was added to assay plates to generate the 11-point dose response curve (final volume 40 μL). Final dilution was ˜1:808 with total solvent concentration ˜0.1%. For combination matrices, 4.5 μL aliquots from dilution plates of orthogonally-titrated master plates were added to the same assay plate to generate the dose-response matrix (final volume of 44 μL). Final dilution of each compound was ˜1:988 with total solvent concentration ˜0.2%. After compound addition, plates were incubated at 37° C./5% CO₂ for 72 hours.

### Day 5: Measure Cell Viability

A solution of 5% CellTiter-Blue (Promega) viability dye in complete medium was dispensed to assay plates using a multi-drop or 384-well pipettor. An appropriate volume was added for a final dye concentration of 2.5%. Viability reactions were incubated for 4-6 hours depending on cell type at 37° C./5% CO₂ to allow reduction of viability dye. Plates were allowed to cool to room temperature for one hour before reading fluorescence intensity at 590 nm after excitation at 540 nm in a Wallac Victor-V plate reader.

TABLE III
Cell Lines, Media and Reagents

| | Source | Catalog # | Lot# |
|---|---|---|---|
| Cell Lines | | | |
| A549 | ATCC | CCL-185 | 3449902 |
| SKMEL-28 | ATCC | HTB-72 | 348832 |
| SKOV-3 | ATCC | HTB-77 | 3898710 |
| Medium and Reagents | | | |
| Base Medium: RPMI-1640‡ | ATCC | 30-2001 | |
| Penicillin/Streptomycin | Cellgro | 30-002-CI | 30002098 |
| Fetal bovine serum | Gibco | 16000-044 | 1127751 |
| Trypsin-EDTA (0.25%) | Cellgro | 25-053-CI | 25053103 |
| L-glutamine | Gibco | 25030-081 | 11150 |
| Celltiter-Blue Viability Dye | Promega | G8081 | 200719 |

‡Base medium is supplement to create complete medium: 10% FBS, Penicillin/Streptomycin (1:100), there is no need to add L-glutamine if ATCC medium is used within 3 months after receipt.

### QC Criteria

### Primary Plate QC Status

cHTS plate formats contain groups of positive and negative intra-plate control wells that are used for automated quality control. All assay plates are assigned an automated QC value by the LIM system following data collection. Automatic quality control calls are made based on the Z-factor calculated using intra-plate controls using a standard factor Z=1−3(_V+_U)/(V−U), where VU are the mean vehicle (treated) and media (untreated) control levels, and _V,_U are the corresponding standard deviation estimates. Z-factor thresholds are empirically set to group plates into three classes: automatically accepted (Z>0.6), automatically rejected (Z<0.4), and undetermined plates that need to be visually evaluated (0.4<z<0.6). Where necessary the QC status of accepted plates may be reassigned to rejected status based on visual inspection of plate quality, transfer controls or other secondary QC criteria. Plates rejected automatically or by visual inspection are excluded from further analysis and scheduled to be repeated.

### Transfer Controls

A positive control compound (Gentian Violet) is included on all master plates. This provides a visual check for screening scientists to verify compound transfer from both column and row masters into the assay plate.

### Secondary QC

Secondary QC includes additional manual checks of data quality including visual inspection of plate quality and transfer controls, marking of data spikes, and checking for cell-line appropriate behavior of single agents. Plates with an accepted status from primary QC that show an unacceptable plate gradient are adjusted to rejected status and queued for repeat. Plates are also visually inspected for occasional bad wells, or “spikes” with data values that are very different from their immediate neighbors (within the same treatment class). These data spikes are flagged in the database, and excluded from subsequent analyses. Finally, dose-response matrices containing single-agent activity inconsistent with past experience will be marked with rejected status and queued for repeat. Data blocks that did not achieve the cut-off threshold were flagged in the database, excluded from subsequent analysis and queued for repeat as necessary.

### Measuring Antiproliferative Activity

The measure of effect was the inhibition of cell viability using an alamar blue viability assay relative to the untreated level (vehicle alone). For untreated and treated levels U and T, a fractional inhibition I=1−T/U was calculated. The inhibition ranges from 0% at the untreated level to 100% when T=0.

Each treated level T was compared to the median untreated level U±σU, determined for each plate by finding the median alamar blue level (and its associated uncertainty, described above) among the untreated control wells arranged across the plate. Applying standard error propagation rules to the expression for I, the estimated standard error σI ˜(σU/U) sqrt(1−I).

The error estimates were further increased to account for variations between replicate combination blocks as well as a minimum assumed fractional uncertainty of _min ˜3%. Thus for inhibition, the standard error estimate becomes σI ˜sqrt{(σU/U)2 (1−I)+σrep 2+σₘᵢₙ².

### Medians and Error Estimates

Medians were used rather than averages to reduce the effect of occasional outliers on the consensus. While medians are more robust to outliers, they are more sensitive to statistical noise, yielding ˜30% larger deviations. Standard deviations are estimated from the median absolute deviation (MAD), where for a normal distribution, the sample deviation σdat ˜1.5 MAD. The standard error for the median itself is then σmed ˜σdat/sqrt(N−1), given N data values.

### Single Agent Dose Curves

The single agent activity is characterized by fitting a sigmoidal function of the form I=Imax/[1+(C/EC₅₀)^{σ}], with least squares minimization using a downhill simplex algorithm. Here, C is the concentration, EC₅₀ is the effective concentration at 50% inhibition, and σ is the sigmoidicity. The uncertainty of each fitted parameter was estimated from the range over which the change in reduced chi-squared χ2 is less than one, or less than minimum reduced χ² if that minimum exceeds one, to allow for underestimated σI errors.

To ensure optimal concentration the EC₅₀ was determined and maximum effect level in each of the proposed proliferation assays. 384-well plates were used to obtain duplicate dose response curves in 12-step dilutions with a dosing ratio f=2, 3, or 4, to cover 3-7 orders of magnitude.

### Selecting Optimal Concentrations

We use the single agent curve data to define a dilution series for each compound to be used for combination screening. Using a dilution factor f of 2, 3 or 4, depending on the sigmoidicity of the single agent curve, we will choose 5 dose levels with the central concentration close to the fitted EC₅₀. For compounds with no detectable single agent activity, we will use f=4 starting from the highest achievable concentration.

### Combination Dose Matrices and Reference Models

The cHTS screening produces dose matrices which contain all pairwise combinations of two single agents at a series of concentrations, including zero. Each dose matrix contains internal copies of the single agent curves which are used as the reference for combination effects. Replicate dose matrices can be merged together by medianing the corresponding data points, and when the concentration series differ, corresponding values are found using bilinear interpolation. Standard errors were computed for each inhibition value using the formulas described above. Combination effects were most readily characterized by comparing each data points inhibition to that of a combination reference model that was derived from the single agent curves. Three models are generally used:

(1) The highest single agent model IHSA(CX,CY)=max(IX,IY) is a simple reference model, where CX,Y are the concentrations of the X and Y compound, and IX,Y are the inhibitions of the single agents at CX,Y;
(2) Bliss independence IBliss(CX,CY)=IX+IY−IXIY represents the statistical expectation for independent competing inhibitors; and
(3) Loewe additivity, where ILoewe(CX,CY) is the inhibition that satisfies (CX/ECX)+(CY/ECY)=1, and ECX,Y are the effective concentrations at ILoewe for the single agent curves.
Loewe additivity is the generally accepted reference for synergy[4], as it represents the combination response generated if X and Y are the same compound. Both IHSA and IBliss are easily calculated from IX,Y, but determining ILoewe requires interpolation and numerical root finding.

### Selecting Combinations for 9×9 Re-Test

To select desirable oncology combinations for repeat assays using high resolution 9×9 dose matrices, three important considerations were evaluated: (1) significant synergy over the additive model; (2) substantial activity where the synergy occurs; and (3) sufficient potency shifting. A “Synergy Score” was used whereby S=log fX log fY_Idata (Idata−ILoewe), summed over all non-single-agent concentration pairs, and where log fX,Y are the natural logarithm of the dilution factors used for each single agent. This effectively calculates a volume between the measured and Loewe additive response surfaces, weighted towards high inhibition and corrected for varying dilution factors. This volume score emphasizes the overall synergistic or antagonistic effect of the combination, thus minimizing the effects of outlying data spikes and identifying combinations with a robust synergy across a wide range of concentrations and at high effect levels. S is positive for mostly synergistic combinations and negative for antagonism. In cases where both synergy and antagonism are present at different concentrations, the weighting favors effects at high inhibition levels. An uncertainty aS is calculated for each synergy score, based on the measured errors for the Idata values and standard error propagation. The synergy score was used and its error to define an appropriate selection cutoff. For example, combinations with S>2_S are significant at ˜95% confidence, assuming a normal distribution. Also, to ensure a sufficient potency shift, the combination index, CI=(CX/ECX)+(CY/ECY) at a chosen effect level is small enough to represent a useful synergy. Observed in vitro CI measurements for currently used clinical combinations (CI ˜0.5-0.7) can be used as a guide in setting the cutoff.

The Table below lists the combinations showing the best synergy with {6-[4-(4-ethyl-piperazin-1-ylmethyl)-phenyl]-7H-pyrrolo[2,3-d]pyrimidin-4-yl]-((R)-1-phenyl-ethyl)-amine.

| | Synergy | Cell |
|---|---|---|
| Combination | Score | Line |
| {6-[4-(4-ethyl-piperazin-1-ylmethyl)-phenyl]-7H-pyrrolo[2,3- | 2.550 | SKOV3 |
| d]pyrimidin-4-yl]-((R)-1-phenyl-ethyl)-amine + N-[1-cyclohexyl-2-oxo- | | |
| 2-(6-phenethyl-octahydro-pyrrolo[2,3-c]pyridin-1-yl-ethyl]-2- | | |
| methylamino-propionamide | | |
| {6-[4-(4-ethyl-piperazin-1-ylmethyl)-phenyl]-7H-pyrrolo[2,3- | 1.957 | A549 |
| d]pyrimidin-4-yl]-((R)-1-phenyl-ethyl)-amine + Cladribine | | |
| {6-[4-(4-ethyl-piperazin-1-ylmethyl)-phenyl]-7H-pyrrolo[2,3- | 1.606 | A549 |
| d]pyrimidin-4-yl]-((R)-1-phenyl-ethyl)-amine + Etoposide | | |
| {6-[4-(4-ethyl-piperazin-1-ylmethyl)-phenyl]-7H-pyrrolo[2,3- | 1.081 | A549 |
| d]pyrimidin-4-yl]-(R)-1-phenyl-ethyl)-amine + Butanedinitrile | | |
| {6-[4-(4-ethyl-piperazin-1-y(methyl)-phenyl]-7H-pyrrolo[2,3- | 1.794 | SKOV3 |
| d]pyrimidin-4-yl]-((R)-1-phenyl-ethyl)-amine + Teniposide | | |
| {6-[4-(4-ethyl-piperazin-1-ylmethyl)-phenyl]-7H-pyrrolo[2,3- | 1.313 | A549 |
| d]pyrimidin-4-yl]-((R)-1-phenyl-ethyl)-annine + Floxuridine | | |
| {6-[4-(4-ethyl-piperazin-1-ylmethyl)-phenyl]-7H-pyrrolo[2,3- | 1.001 | A549 |
| d]pyrimidin-4-yl]-((R)-1-phenyl-ethyl)-amine + Staurosporine | | |
| {6-[4-(4-ethyl-piperazin-1-ylmethyl)-phenyl]-7H-pyrrolo[2,3- | 1.069 | SKMEL28 |
| d]pyrimidin-4-yl]-((R)-1-phenyl-ethyl)-amine + Mitoxantrone | | |
| Hydrochloride | | |

1-5. (canceled)

6. A combination of:
(a) a compound of the formula (I): 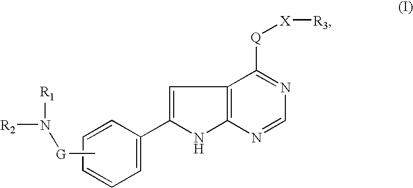
wherein
R₁, and R₂ are each independently of the other hydrogen, unsubstituted or substituted alkyl or cycloalkyl, a heterocyclic radical bonded via a ring carbon atom, or a radical of the formula R₄—Y—(C=Z)-, wherein R₄ is unsubstituted, mono- or di-substituted amino or a heterocyclic radical, Y is either not present or lower alkyl and Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇-alkylene, —C(═O)— or C₁-C₆-alkylene-C(═O)— wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a salt thereof; and
(b) one or more pharmaceutically active agents selected from the group consisting of N-[1-cyclohexyl-2-oxo-2-(6-phenethyl-octahydro-pyrrolo[2,3-c]pyridin-1-yl-ethyl]-2-methylamino-propionamide, floxuridine, cladribine, butanedinitrile, staurosporine; teniposide; etoposide; for simultaneous, concurrent, separate or sequential use in for preventing or treating a proliferative disease.

7. (canceled)

8. A method of preventing or treating a proliferative disease comprising the combination according to claim 6.

9. The method of claim 8, wherein the proliferative disease is selected from ovarian cancer, lung carcinoma and melanoma.

10-14. (canceled)

15. A pharmaceutical composition comprising:
(a) A compound of the formula (I): 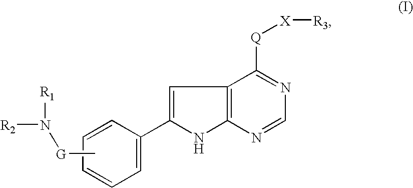
wherein
R₁, and R₂ are each independently of the other hydrogen, unsubstituted or substituted alkyl or cycloalkyl, a heterocyclic radical bonded via a ring carbon atom, or a radical of the formula R₄—Y—(C=Z)-, wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇-alkylene, —C(═O)—, or C₁-C₆-alkylene-C(═O)— wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a salt thereof; and
(b) one or more pharmaceutically active agents selected from the group consisting of N-[1-cyclohexyl-2-oxo-2-(6-phenethyl-octahydro-pyrrolo[2,3-c]pyridin-1-yl-ethyl]-2-methylamino-propionamide, floxuridine, cladribine, butanedinitrile, staurosporine; teniposide; etoposide and a mixture thereof.

16. (canceled)

17. A method of preventing or treating a proliferative disease comprising the combination according to claim 15.

18. The method of claim 17, wherein the proliferative disease is selected from ovarian cancer, lung carcinoma and melanoma.

19-22. (canceled)

23. A method of treating a proliferative disease comprising a combination of:
(a) a compound of the formula (I): 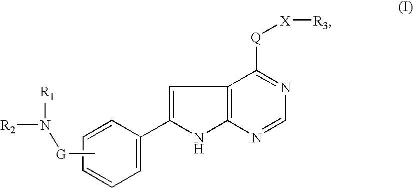
wherein
R₁ and R₂ are each independently of the other hydrogen, unsubstituted or substituted alkyl or cycloalkyl, a heterocyclic radical bonded via a ring carbon atom, or a radical of the formula R₄—Y—(C=Z)- wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen, or
R₁, and R₂, together with the nitrogen atom to which they are attached, form a heterocyclic radical;
R₃ is a heterocyclic radical or an unsubstituted or substituted aromatic radical;
G is C₁-C₇-alkylene, —C(═O)— or C₁-C₆-alkylene-C(═O)—, wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a salt thereof; and
(b) one or more pharmaceutically active agents selected from the group consisting of N-[1-cyclohexyl-2-oxo-2-(6-phenethyl-octahydro-pyrrolo[2,3-c]pyridin-1-yl-ethyl]-2-methylamino-propionamide, floxuridine, cladribine, butanedinitrile, staurosporine; teniposide; etoposide and a mixture thereof.

24. (canceled)

25. The method according to claim 23, wherein the proliferative disease is selected from ovarian cancer, lung carcinoma and melanoma.

26-31. (canceled)

32. A commercial package comprising:
(a) a pharmaceutical composition comprising a compound of the formula (I): 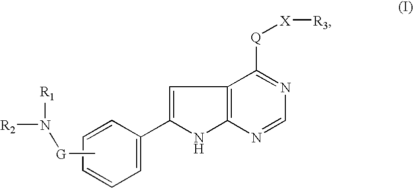
wherein
R₁ and R₂ are each independently of the other hydrogen, unsubstituted or substituted alkyl or cycloalkyl, a heterocyclic radical bonded via a ring carbon atom, or a radical of the formula R₄—Y—(C=Z)-, wherein R₄ is unsubstituted, mono- or disubstituted amino or a heterocyclic radical, Y is either not present or lower alkyl and Z is oxygen sulfur or imino, with the proviso that R₁ and R₂ are not both hydrogen, or
R₁ and R₂, together with the nitrogen atom to which they are attached, form a heterocyclic radical;
R₃ is a heterocyclic radical or an unsubstituted or substituted aromatic radical;
G is C₁-C₇-alkylene, —C(═O)—, or C₁-C₆-alkylene-C(═O)— wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a salt thereof; and
(b) a pharmaceutical compositions of a pharmaceutically active agent compound selected from the group consisting of N-[1-cyclohexyl-2-oxo-2-(6-phenethyl-octahydro-pyrrolo[2,3-c]pyridin-1-yl-ethyl]-2-methylamino-propionamide, floxuridine, prednisone, cytarabine, cladribine, butanedinitrile, staurosporine, teniposide, mitoxantrone hydrochloride, etoposide and a mixture thereof;
wherein (a) and (b) are administered together, one after the other or separately in one combined unit dosage form or in two separate unit dosage forms.

33. The commercial package according to claim 32, wherein the unit dosage form is a fixed combination.

34. A method of preventing or treating a proliferative disease comprising the combination according to claim 32.

35. The method of claim 34, wherein the proliferative disease is selected from ovarian cancer, lung carcinoma and melanoma.

36. The combination according to claim 6, where the compound of the formula (1) is {6-[4-(4-ethyl-piperazin-1-ylmethyl)-phenyl]-7H-pyrrolo[2,3-d]pyrimidin-4-yl}-((R)-1-phenyl-ethyl)-amine.

37. The pharmaceutical composition according to claim 15, where the compound of the formula (I) is {6-[4-(4-ethyl-piperazin-1-ylmethyl)-phenyl]-7H-pyrrolo[2,3-d]pyrimidin-4-yl}-((R)-1-phenyl-ethyl)-amine claim 38. The method according to claim 23, where the compound of the formula (I) is {6-[4-(4-ethyl-piperazin-1-ylmethyl)-phenyl]-7H-pyrrolo[2,3-d]pyrimidin-4-yl}-((R)-1-phenyl-ethyl)-amine.

39. The commercial package according to claim 32 where the compound of the formula (I) is {6-[4-(4-ethyl-piperazin-1-ylmethyl)-phenyl]-7H-pyrrolo[2,3-d]pyrimidin-4-yl}-((R)-1-phenyl-ethyl)-amine.
